(19) United States
(12) Patent Application Publication
(10) Pub. No.: US 2011/0112149 A1
(43) Pub. Date: May 12, 2011

(54) EPOTHILONE ANALOGUES, THEIR PHARMACEUTICAL COMPOSITIONS, THEIR USE AND THEIR PREPA RATIONS

(76) Inventors: Li Tang, Beijing (CN); Rongguo Qiu, Beijing (CN)

(21) Appl. No.: 12/919,487

(22) PCT Filed: Mar. 2, 2009
(86) PCT No.: PCT/CN2009/000218

§ 371 Date: Nov. 30, 2010

(30) Foreign Application Priority Data
Feb. 29, 2008 (CN) 200810082360.5

(51) Int. Cl. A61K 31/427; C07D 417/06; C07D 493/04; C07D 401/06; A61K 31/4427; A61P 35/00; A61P 25/00; A61P 19/02; A61P 9/10

(52) U.S. Cl. 514/340; 548/204; 540/463; 548/159; 514/365

(57) ABSTRACT

The present invention relates to novel 15-membered thiazole lactone or lactam polyketide compounds, their pharmaceutical compositions, their use and their preparations. The disclosed compounds relate to those of general formula I, their preparations and their use for preparing therapeutical compositions used as cell inhibitors.

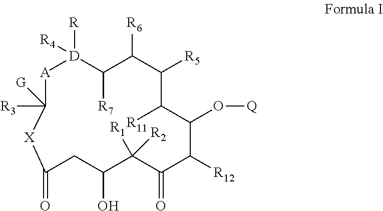

### FIELD OF THE INVENTION

The present invention relates to novel 15-membered polyketide compounds and intermediates thereof, said polyketide compounds may refer to the derivatives of natural epothilone in terms of their structure. The present invention also relates to their preparations and their pharmaceutical uses, particularly their uses in the preparation of a pharmaceutical composition comprising the same.

### BACKGROUND OF THE INVENTION

Epothilone A (EpoA) and Epothilone B (EpoB) are 16-membered ring thiazole macrolide compounds, first had been isolated from soil bacteria, Sorangium cellulosum strain So ce90, having the structure below [Hofle et. al., 1996, Angew. Chem. Int. Ed. Engl. 35(13/14): 1567-1569; Gerth et. al., 1996. J. Antibiotics 49(6): 560-563].

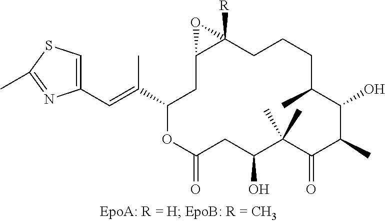

Epothilone has a great potential in the treatment of cancers. Although their structures are different, the action mechanism of epothilones is quite similar to the well-known anti-tumor agent of paclitaxel (Taxol), including inducement of microtubule polymerization and microtubule stabilization. Those compounds exhibit powerful killing-capability on different tumor cell lines. Specifically, they exhibit remarkable effects on several multidrug-resistant tumor cell lines, especially paclitaxel-resistant tumor cell lines or tumor cell lines having resistance to some other anti-tumor drugs [Altmann et. al., 2000. Biochem. Biophys. Acta. 1470(3): M79-91; Bollag et. al., Cancer Res. 55(11): 2325-2333].

Epothilone C and D, the desoxy-counterparts of epothilones A and B, have been successfully synthesized via chemical total synthesis, which, however, can also be detected along with other trace components having Epothilone-like structures in the fermentation extracts of natural epothilone producing strain, S. cellulosum. At present, attentions have been focused on the development of more effective Epothilone chemotherapeutants and analogues of Epothilone with related structures. For example, the naturally-occurring Epothilone compounds may be modified by chemical semi-synthesis, such as a reaction converting Epothilone B into the corresponding lactam analogue BMS247550, as described in WO99/27890.

It has been found that the 15-membered thiazole polyketide lactone or lactam compounds related to epothilones have beneficial pharmacologic properties, and can be used in the treatment of proliferative diseases. There has been a long-lasting interest on novel epothilone derivatives.

### DISCLOSURE OF THE INVENTION

The objective of the present invention is to provide a series of 15-membered thiazole polyketide lactone or lactam derivatives; and to provide a method for preparing the same, by which such novel compounds of the present invention and their novel derivatives can be obtained from compounds such as Epothilone D or B and 4-demethylated Epothilone D or B via chemical synthesis or chemical modifications and bioconversion and the like. The present invention further provides the use of such novel thiazole polyketide lactone or lactam compounds for the preparation of pharmaceutical compositions used for anti-tumor, inhibiting excessive cell growth and terminating cell growth.

Polyketide compound according to the present invention, i.e. a novel 15-membered thiazole polyketide compound is represented by the following general formula I

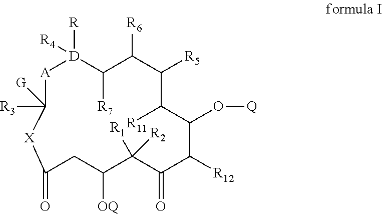

wherein,

As A-D represents a C═C bond of formula (a) or an epoxy group of formula (b) below, R₄

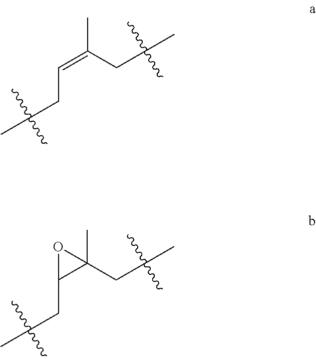

is not exist,

As A-D represents a C—C bond, R₄ represents a hydroxy group or H,

G is selected from a substituted or unsubstituted alkyl group, a substituted or unsubstituted aryl group, a heteroaryl group, a heterocyclic group, a cycloalkyl group, or any one selected from the following formulae:

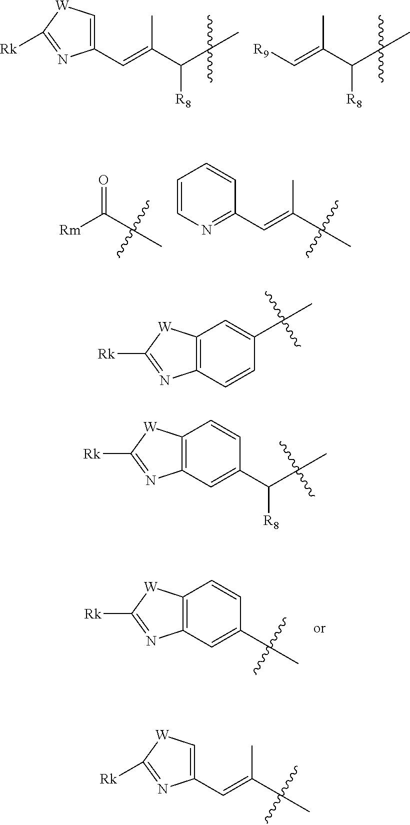

Q is selected from H, a C₁₋₄ alkyl group, NH₂ or a hydroxy-protecting group such as silyl ether selected from any one of TMS, TES or TBS:

R₁, R₂ are each independently selected from H or a substituted or unsubstituted alky group (preferably a C₁₋₄ alkyl group, more preferably methyl group), or together form a cycloalkyl group;

R₈ is selected from H, a hydroxy group, a substituted or unsubstituted alky group (preferably a C₁₋₄ alkyl group, more preferably methyl group) or NH₂, N₃ or NR₁₃R₁₄;

X represents O, S or N—R₁₅, wherein R₁₅ represents H, NR₁₆R₁₇, a substituted or unsubstituted alky group (preferably a C₁₋₄ alkyl group, more preferably methyl group), a substituted or unsubstituted aryl group, a cycloalkyl group or a heterocyclic group;

Rm is selected from H, methyl, NR₁₆R₁₇ or halomethyl;

R₁₂ is selected from H, an allyl group, a hydroxy group, NH₂ or a substituted or unsubstituted alky group (preferably a C₁₋₄ alkyl group, more preferably methyl group); preferably, R₁₂ is an allyl group;

R₉ is selected from H, a substituted or unsubstituted alky group (preferably a C₁₋₄ alkyl group, more preferably methyl group), aryl group, a heteroaryl group, a cycloalkyl group or a heterocyclic group, said heteroaryl group is preferably selected from thiazolyl, pyridyl, oxazolyl, isoxazolyl, quinoline or benzoxazolyl;

R₃, R₄, R₅, R₆, R₇, R₁₁, R₁₃, R₁₄, R₁₆, R₁₇ are each independently selected from H, a hydroxy group, NH₂ or a substituted or unsubstituted alky group (preferably a C₁₋₄ alkyl group, more preferably methyl group), wherein R₅, R₆ may also together form a C═C bond;

Rk is selected from H, methyl, a substituted or unsubstituted alky group (preferably a C₁₋₄ alkyl group, more preferably methyl group), an aminoalkyl group, a hydroxyalkyl group or a haloalkyl;

R is selected from H, trifluoromethyl, a substituted or unsubstituted alky group (preferably a C₁₋₄ alkyl group, more preferably methyl group) or halogen;

W represents S or O, NH, N-alkyl;

or a pharmaceutical acceptable salt, hydrate, polymorph, optical isomer, racemate, diastereomer or enantiomer of said compound.

Preferably, G in compound of formula I is selected from:

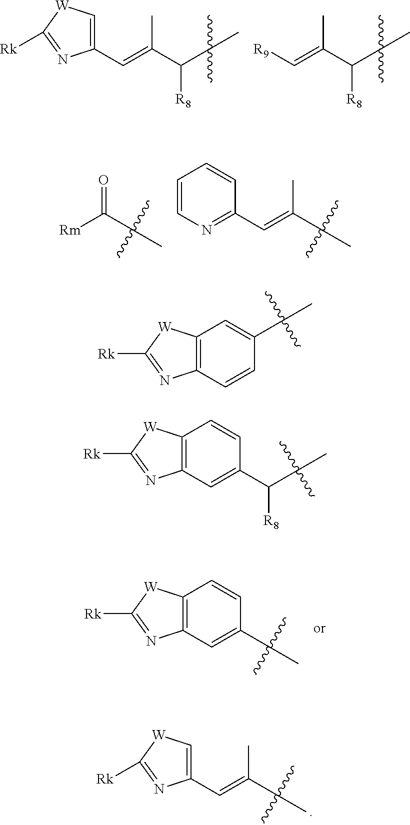

In one embodiment, the compound of the present invention is represented by the structure of the following general formula II:

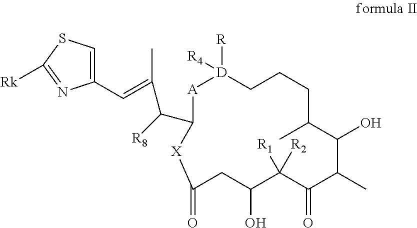

wherein, X is NR₁₅ or O;

R₈ is NHR₁₅ or OQ; each of the other symbols as given has the same meaning as defined above.

In another embodiment, the compound of the present invention is represented by the structure of the following general formula III:

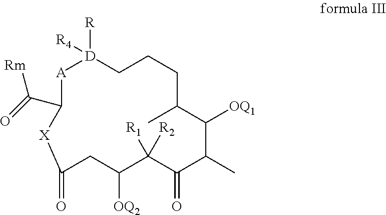

wherein, Q₁ and Q₂ each represents H, a C₁₋₄ alkyl group, NH₂ or hydroxy-protecting group such as silyl ether selected from any one of TMS, TES or TBS; each of the other symbols has the same meaning as defined above.

In another embodiment, the compound of the present invention is represented by the structure of the following general formula IV:

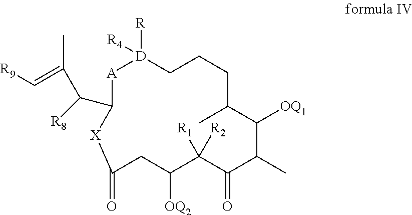

wherein, each symbol in formula IV has the same meaning as defined above.

In another embodiment, compound of the present invention is represented by the structure of the following general formula V:

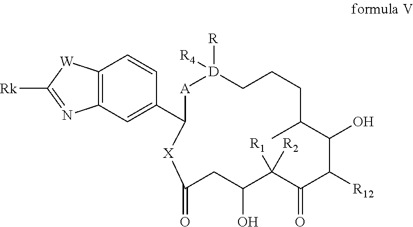

wherein, X is NR₁₅ or O; R₁₅ is H, a methoxy group or an alkyl group; R₁₂ is H, an allyl group, a substituted or unsubstituted alkyl group (preferably a C₁₋₆ alkyl group, more preferably methyl group)

In still another embodiment, compound of the present invention is represented by the structure of the following general formula VI:

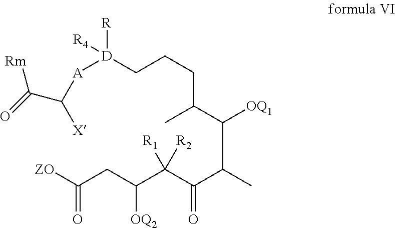

wherein

X′ is NHR₁₅, NR₁₅P, OH or OQ; wherein, R₁₅ is H, a methoxy group or an alkyl group; P is a N-protecting group;

Z is H, a substituted or unsubstituted alkyl group (preferably a C₁₋₆ alkyl group, more preferably methyl, tert-butyl), a cycloalkyl group, an aryl group or a carboxyl-protecting group;

Q₁ and Q₂ each independently represents H, a C₁₋₄ alkyl group, NH₂ or a hydroxy-protecting group.

Unless otherwise indicated, the terms as used herein are of the following meanings:

Any asymmetric carbon atoms may be in the configuration of (R)—, (S)— or (R, S)—, therefore, the compound of the present invention may present in a large amount of variety form of optical isomers, geometrical isomers and stereoisomers, and that all these isomers and mixtures thereof are included in the scope of the present invention.

Unless otherwise indicated, alkoxy, alkyl (including the alkyl of hydroxyalkyl, haloalkyl and aminoalkyl) defined in the compound of the present invention preferably refers to a lower alkyl or a lower alkoxy. The term of “lower” refers to a group having 6 or less carbon atoms, preferably a group having 4 or less carbon atoms, wherein said group may be a straight chain or a branched chain having one or more branches. Substituted alkyl group refers to an alkyl group substituted by 1 to 4 substituents, wherein said substituent may be a halogen, trifluoromethyl, trifluoromethoxy, alkylacyl, aryloxy, amino, alkylamino, heterocyclic amino, arylamino, arylalkylamino, alkylacylamino, alkylsulfonyl, alkythio, alkoxycarboxyl etc. An aryl group refers to a monocyclic or bicyclic aryl group having 6-12 carbon atoms on the ring, such as phenyl, diphenyl etc. The aryl group may be a substituted or unsubstituted aryl group. The substituted aryl group refers to an aryl group substituted by 1 to 4 substituents, wherein said substituent may be, for example, a substituted or unsubstituted alkyl group, a halogen, CF₃, trifluoromethoxy, hydroxy, alkoxy, cycloalkoxy, cycloalkylamino, alkylacyl, aryloxy, amino, alkylamino, heterocyclic amino, arylamino, arylalkylamino, alkylacylamino, alkylsulfonyl, mercapto, alkythio, cycloalkythio, nitro, carboxyl, carboxyalkyl, carbamyl, alkoxycarboxyl etc. Heteroaryl group refers to any 5-membered or 6-membered ring having 1 to 3 heteroatoms selected from nitrogen, oxygen and/or sulfur, wherein 5-membered ring has 0 to 2 double bonds, while 6-membered ring has 0 to 3 double bonds. The Heteroatom of nitrogen or sulfur may be optionally oxidized, and also optionally quaternized. The monocyclic or bicyclic unsaturated heteroaryl group having at least one nitrogen atom and 0 or 1 oxygen atom and 0 or 1 sulfur atom is preferable, and a group having 5 to 12, more preferably 5 or 6, ring atoms on the connecting ring is more preferable, and that said heteroaryl group may be unsubstituted or substituted by one or more substituents preferably selected from halogen, alkoxy, alkythio, hydroxy, alkyl and/or alkylacyl. Heteroaryl group is preferably selected from thiazolyl, pyridyl, oxazolyl, quinoline or benzoxazolyl or benzothiazolyl. Cycloalkyl group refers to a saturated carbon ring that may be optionally substituted, having preferably 1 to 3 rings, and 3 to 7 carbon atoms on each ring, which may fuse together with an unsaturated C₃-C₇ carbon ring. Cycloalkyl group is preferably cyclopropyl, cyclopentyl etc. Heterocyclic group refers to a saturated non-aromatic cyclic group that may be optionally substituted, such as 4- to 7-membered monocyclic ring, 7- to 11-membered bicyclic ring, or 10- to 15-membered tricyclic ring, and that at least one heteroatom is present on at least one of the rings, and 1, 2, or 3 heteroatoms selected from nitrogen, oxygen and sulfur may be present in the ring of each heterocyclic group, wherein the heteroatom of nitrogen and sulfur may be optionally oxidized, and nitrogen atom may also be optionally quaternized. The heterocyclic group may be linked at any heteroatomes or carbon atoms. Heteroaryl group may be fused with an unsaturated C₃-C₇ carbon ring, which results in the formation of groups, for example, pyazolidyl, thiazolyl, pyridyl, oxazolyl, isoxazolyl, quinoline, benzoxazolyl or benzothiazolyl, imidazolyl and furanyl.

The hydroxy-protecting group, N-protecting group and carboxyl-protecting group used in the compounds of the present invention refer to the protecting group commonly employed in this art, for example, hydroxy-protecting group is preferably silyl ether, such as TMS, TES or TBS; N-protecting group is preferably tert-butylcarboxylate; carboxyl-protecting group is preferably methyl, tert-butyl (e.g. tert-butyl formed from tert-butanol and carbodiimide) and the like.

During the process for preparing the compound of the present invention, functional groups in the starting compounds that do not involve in the reaction may be unprotected dependent on the particular process step to be taken place, or they may be protected by one or more protecting groups, or completely or partly removed. The protecting groups are characterized in that they get easily to be removed by solvolysis, reduction, and photolysis or by enzyme activity, and that they do not remain in the end products. Hydroxy-protecting group is preferably a lower alkylsilyl hydroxy-protecting group as described herein, and they can be introduced and removed in the same manner as the method described herein. Meanwhile, selective protection or deprotection is also possible. Herein, protecting groups are not mentioned in some places to be suitably used, however, a person skilled in the art may realize the suitable time or way for use of the protecting group.

Molecular cyclization may be performed under conventional conditions. For example, if X′ is a hydroxy group, cyclization refers to macrocyclic lactonization, and if X′ is NH₂ or NHR (alkyl), cyclization refers to macrocyclic lactamization. Macrocyclic lactonization takes place in a suitable solvent or solvent mixture from precursor of acid (or anhydride) or from protected derivatives with free hydroxy group under the condition as described in M. Yamaguchi et. al., Bull. Chem. Soc. Jpn. 1979, 52:1989. Lactamization (macrocyclic lactamization) is performed under the conventional condition for reacting carboxylic acid and amide, particularly using a standard coupling agent such as DCC/HOBt or diphenylphosphoryl azide or bromo-tris-pyrrolidino-phosphonium hexafluorophosphate (PyBroP) commonly employed in peptide chemistry for the conversion of a macrocyclic lactam.

Compounds of formula I, where A-D is an epoxy with C—C bond, can be prepared from compound of formula I with A-D being a C═C bond by known method in this art, for example, by using peroxide, preferably dioxirane in a suitable solvent or solvent mixture at a relatively lower temperature.

A preferable compound involves in some embodiments of the present invention, when G is of the following,

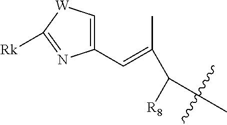

a compound represented by the structure of the following formula II and salts thereof are provided:

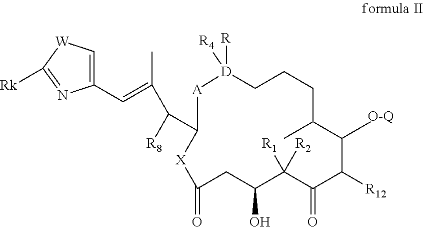

wherein, the compound of general formula II can be prepared from Epothilone or derivatives thereof LL by chemical modification or/and bioconversion, such as synthetic route of reaction 1:

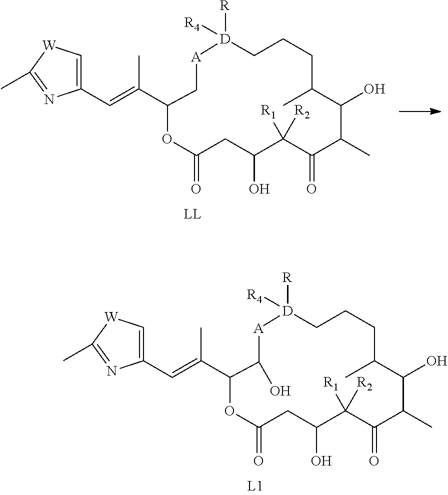

Firstly, C-14 of epothilone or derivatives thereof is hydroxylated by using hydroxylated enzyme derived from microorganisms, for example, by synthetic route of reaction 1. Such a C-14 hydroxylated epothilone derivative L1 can be prepared by microbial conversion as described in Example 1. According to the description disclosed in CN1629283 (published on Jun. 22, 2005), Epothilone C or D can be easily obtained. For example, Epothilone C or D is produced as the main metabolite from the mutant of natural epothilone A and B producing strain by the inactivation of the P450 gene of Epothilone biosynthetic gene. According to the description disclosed in CN1521258 (published on Aug. 18, 2004), 4-demethylated Epothilone A and B or C and D can be easily obtained. Under general circumstances, inactivation of MT domain (methyl-transferring domain) in extender module 8 of Epothilone biosynthetic gene results in the production of 4-demethylated Epothilone as the main metabolite from the mutant of natural epothilone producing strain. These two patent documents are incorporated herein by reference. On the other hand, the present invention relates to a compound of structural formula II, wherein W is O, synthesized from the oxazole counterpart of Epothilone derivative. According to the description of CN1521258, by introducing an excess amount of serine to the epothilone producing strain, the producing strain normally for the production of the thiazole Epothilone compound can be adjusted in a manner that facilitates the production of the oxazole counterpart.

Compound L4, a preferable compound of general formula II can be prepared from C-14 hydroxylated Epothilone derivatives according to the general procedure described in synthetic route of reaction 2. Such compound L4 can be prepared according to the procedure described in Example 2.

Synthetic route of reaction 2:

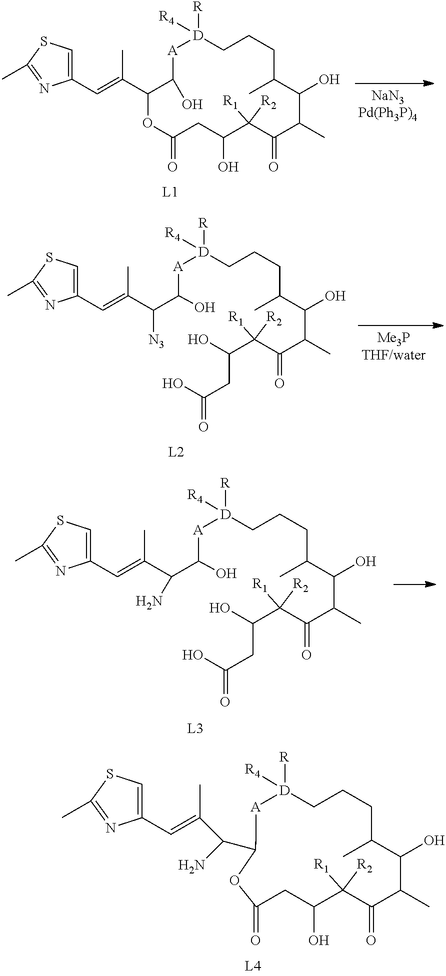

1. Allyl palladium π complex is formed by reacting 14-OH Epothilone and its derivatives with tetrakis(triphenyphosphine)palladium in THF/water, which is then treated with sodium azide, and results in the formation of a ring-opened azide compound.

2. Next, azide is reduced to NH₂ by using Adam's catalyst (PtO₂) or reducing agent such as triphenylphosphine in ethanol.

3. Carboxylic acid and 14-OH in L3 are subjected to macrocyclic lactonization. Under Yamaguchi condition, hydroxy acid is treated with macrocyclic lactonization agent, such as 1,3,5-trichlorobenzoyl chloride, and triethylamine in THF at 0° C., followed by addition of a solution of 4-(dimethylamino)pyridine in toluene to the reacting mixture, and the temperature is elevated to 75° C., and compound L4 or intermediate (protected L4) is obtained.

4. Protecting group may be present in the 14-OH Epothilone precursor, and to protect those relevant functional groups so as to prevent any undesirable side reactions, such as acylation, etherification, oxidation, solvolysis and the like. Protecting group is characterized in that they get easily to be removed by solvolysis, reduction, and photolysis or by enzyme activity, and that they do not remain in the end products. For example, firstly, protecting group can be present in the 3-, 7-, and 14-OH free hydroxy groups of the 14-OH Epothilone precursor as P1, P2 and P3, respectively. Before macrocyclic lactonization, protecting group P3 of 14-OH can be selectively removed by AcOH in THF under the condition that protecting groups P1, P2 are not removed. Finally, carboxylic acid can react with 14-OH undergoing macrocyclic lactonication to obtain the intermediate, protected L4. P1 and P2 can be removed by any means commonly employed in the art. In the case P1 and P2 is silyl ether, such as TMS, TES or TBS, deprotection can be performed by treating with acid, such as HF in dichloromethane, pyridine or trifluoroacetic acid and to afford L4.

Synthetic route of reaction 2-B:

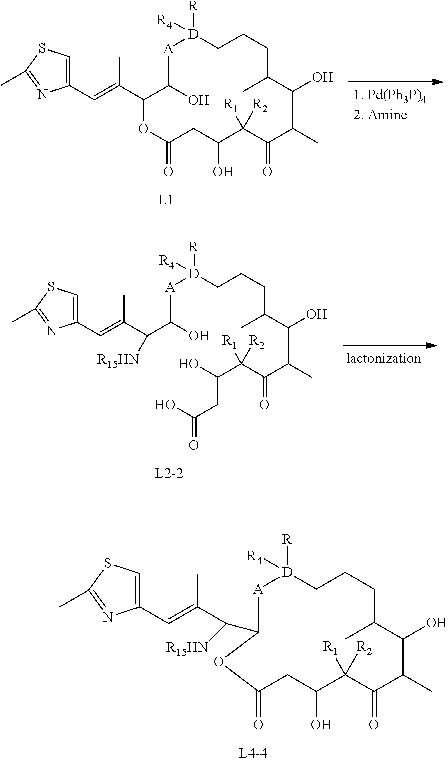

In the case R8 in general formula II is NHR₁₅ and R₁₅ is not H, compound of general formula II can be prepared from C-14 hydroxylated Epothilone derivatives according to the general procedure described in synthetic route of reaction 2-B.

1. Firstly, allyl palladium π complex is formed by using tetrakis(triphenyphosphine)palladium, and then treated with primary amine to afford an intermediate with R15NH—, wherein R15 is OH, a substituted or unsubstituted alkyl group, a cycloalkyl group, an aryl group, a heteroaryl group, O-alkyl.

2. According to the condition described in M. Yamaguchi et. al., Bull. Chem. Soc. Jpn. 1979, 52:1989, macrocyclic lactonization is performed.

3. If R15 is OH, protecting group has to be removed from R15-O-TMS to afford the end product.

Compound L7, a preferable compound of general formula II can be prepared from C-14 hydroxylated Epothilone compound according to the general procedure described in synthetic route of reaction 3, as described in Example 3.

Synthetic route of reaction 3:

1. Firstly, free hydroxy group, 3- and 7-OH, can be protected by a suitable group,

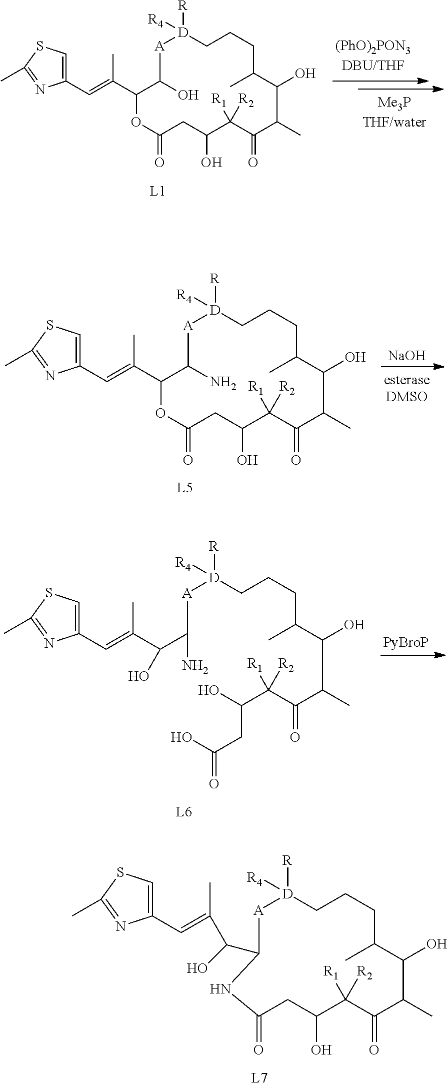

such as triethylsilyl, butyldimethylsilyl etc., respectively as P1 and P2. Protecting group P3 of 14-OH can be selectively removed by using AcOH in THF under the condition that protecting groups P1 and P2 are not removed.

2. Treating the 3,7-protected Epothilone derivative with tetrakis(triphenyphosphine)palladium to form the allyl palladium π complex, followed by treating with primary amine, or reacting with diphenylphosphoryl azide and diazabicyclo(5.4.0)undec-7-ene (DBU) to afford 14-azide Epothilone. Next, reduction is performed by using trimethylphosphine and NH4OH aqueous solution to afford 3,7-protected 14-amino-Epothilone derivative.

3. Under the condition that allows the selective reduction of lactone carboxyl, 3,7-protected 14-amino-Epothilone derivative is reacted with a reducing agent such as di(isobutyl)aluminium hydride (DiBAI-H) to afford the protected ring-opened intermediate.

4. Corresponding macrocyclic lactamization of the protected ring-opened intermediate is performed using a standard amide coupling agent such as diphenylphosphoryl azide and sodium hydrogen carbonate or EDC/HOBT (1-hydroxybenzotriazole) and 1-(3-dimethylaminopropyl)-3-ethylcarbodiimide in DMF or bromo-tris-pyrrolidino-phosphonium hexafluorophosphate (PyBroP) to afford the intermediate, protected L7. Deprotection is performed to afford L7 compound.

Compound L9, a preferable compound of general formula II can be prepared from C-14 hydroxylated Epothilone compound according to the general procedure described in synthetic route of reaction 4.

Synthetic route of reaction 4:

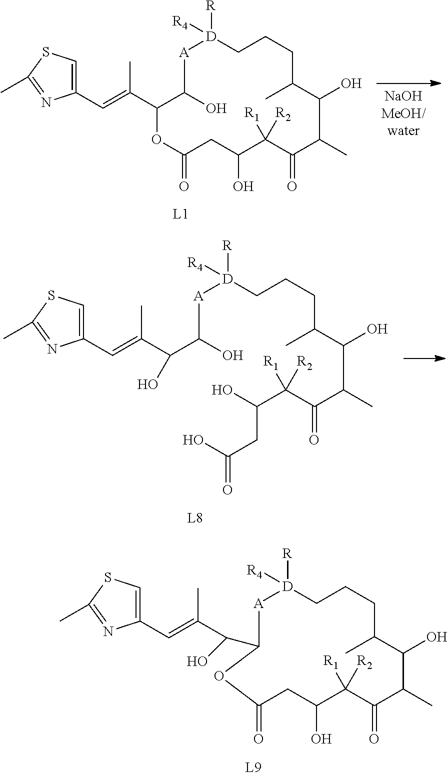

Make reference to synthetic route of reaction 4, the corresponding ring-opened Epothilone hydroxy acid intermediate prepared by saponification is used to prepare compound L9 of the present invention. For example, Epothilone derivatives are converted to a ring-opened seco-acid, hydroxy acid by hydrolysis by treating with a solution of sodium hydroxide in methanol/water or esterase (such as Pig liver esterase) in DMSO. Finally, 14-OH and carboxylic acid are subjected to lactonization according to the method disclosed by Yamaguchi et. al. to afford L9. Technically, the ring-opened Epothilone hydroxy acid intermediate may be converted to tert-butyl ester using tert-butanol, carbodiimide (such as dicyclohexyl carbodiimide) and 4-(dimethylamino)pyridine as catalyst, or said hydroxy acid may be converted to methyl ester by reacting with trimethylsilyl diazomethane. For example, 15-OH of the methyl ester can be protected by silylation, for example, by treating with trimethylsilyl chloride/trimethylsilyl imidazole. The protecting group of butyl ester or methyl ester in the intermediate can be removed by treating with camphorsulfonic acid (CSA) in methanol and dichloromethane or with sodium hydroxide, followed by treating with acetic acid. After subjecting 14-OH and carboxylic acid to macrocyclic lactonization, intermediate, i.e. L9 having the free hydroxy group protected is obtained, and then L9 is obtained after deprotection.

A preferable compound involves in some embodiments of the present invention, when G is of the following,

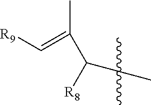

a compound represented by the structure of the following formula III and salts thereof are provided:

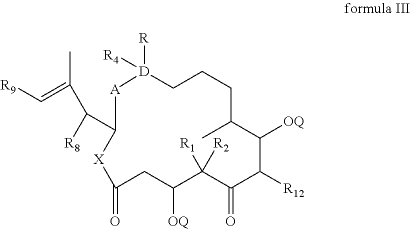

Compound of general formula III can also be prepared from Epothilone D or derivatives thereof such as 4-demethylated Epothilone D according to the general procedure described in synthetic route of reaction 5, as described in Example 4.

Synthetic route of reaction 5:

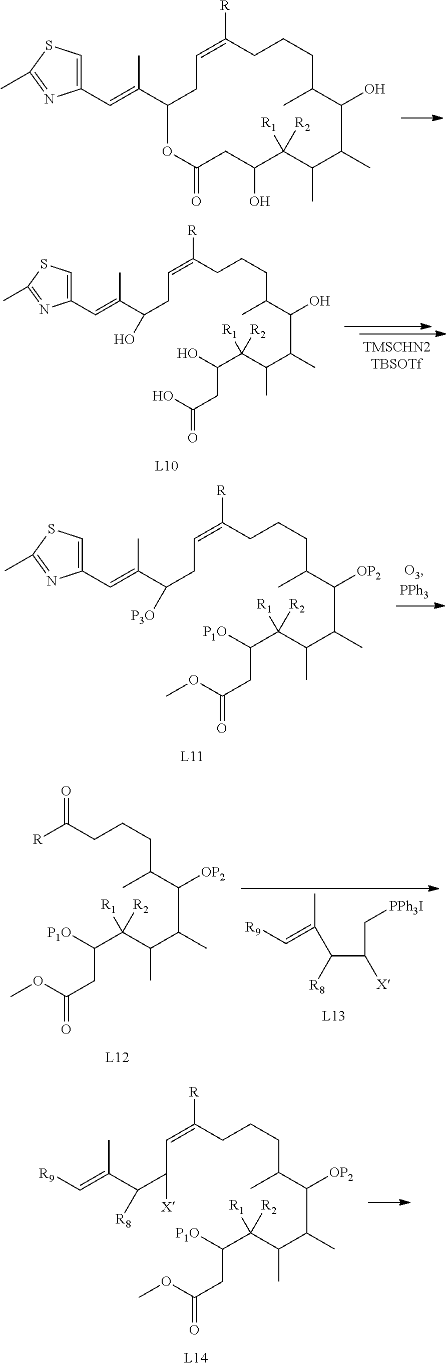

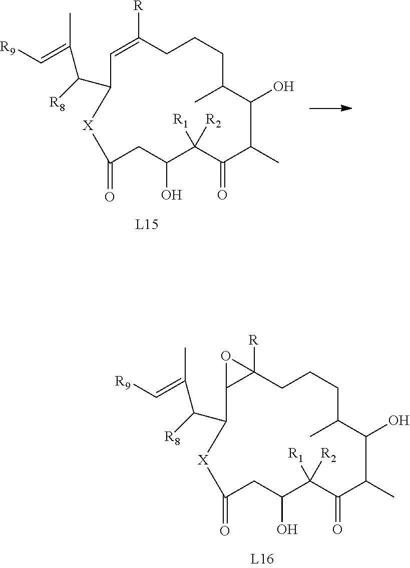

wherein, X′ is OP3, NR15P or SP', P1 and P2 each independently represents H or the same or different protecting group, P3 is H or a protecting group, P is H or N-protecting group, P′ is H or S-protecting group. X is O, NH, NR₁₅ or S.

1. The corresponding ring-opened Epothilone hydroxy acid intermediate prepared by saponification is used. For example, Epothilone derivative is converted to a ring-opened seco-acid, hydroxy acid by subjecting it to hydrolysis by treating with an aqueous solution of sodium hydroxide in methanol/water or with a suitable esterase (e.g. Pig liver esterase etc).

2. The ring-opened Epothilone hydroxy acid intermediate can be converted to methyl ester by reacting with an alkylating agent such as trimethylsilyl diazomethane (TMSCHN₂). 3-, 7- and 15-free hydroxy group of methyl ester can be protected (P1, P2 or/and P3), for example, by silylation by treating with trimethylsilyl chloride/trimethylsilyl imidazole, and t-butyldimethylsilyl trifluoromethane sulfonate to introduce the same or different protecting groups.

3. Double bond at C12 in protected L11 is subjected to oxidative cleavage by using ozone to afford compound L12.

4. L12 and compound L13 are subjected to Wittig olefination to afford L14.

5. Protecting group of methyl ester can be removed by treating with a particular type of base such as hydroxide (LiOH), and to afford a carboxylated ring-opened Epothilone derivative.

The selectively deprotected 14-X′ and 1-carboxylic acid are subjected to macrocyclic lactonization or lactamization, to afford L15 with 3-, 7-protected or deprotected.

6. Finally, vinyl group of L15 is subjected to epoxidation to afford L16.

A preferable compound involves in some embodiments of the present invention, when G is of the following

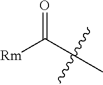

a compound represented by the structure of the following formula IV and salts thereof are provided:

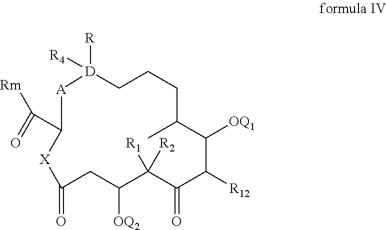

Synthetic route of reaction 6:

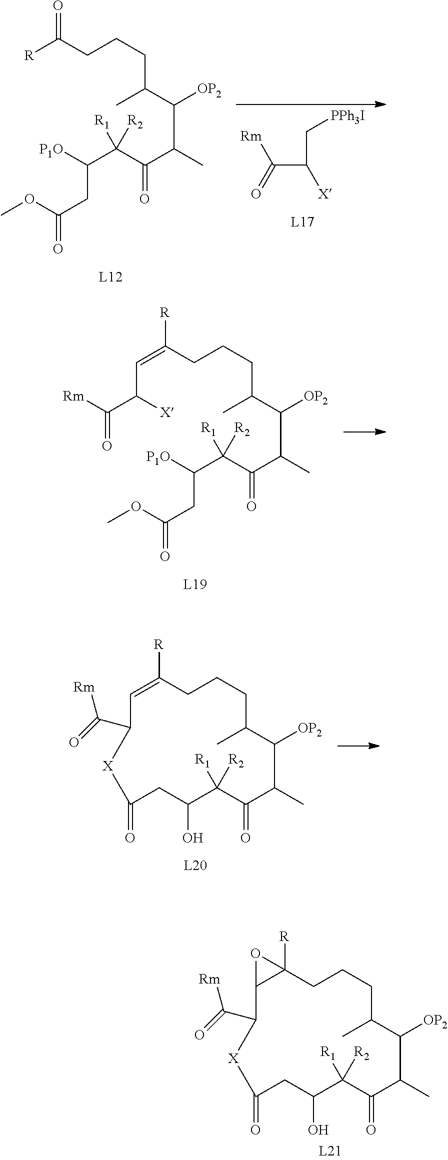

Compound of general formula IV can also be prepared from L12 and L17 (preferably Rm=CH₃) according to the general procedure described in synthetic route of reaction 6. The synthetic procedure is equivalent to the general procedure 4, 5, 6 described in synthetic route of reaction 5.

Synthetic route of reaction 7:

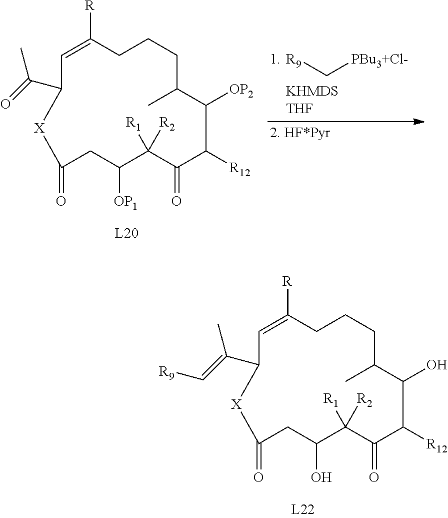

In other embodiments of the present invention, compound L22 can be prepared from compound of formula IV according to the synthetic procedure described in synthetic route of reaction 7. See example 5, wherein R9 is preferably

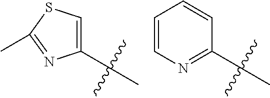

For example, as described in A. Rivkin et. al., J. Am. Chem. Soc. 2003, 125:2899, ketone compound L20, where P1 and P2 is a hydroxy-protecting group, reacts with a suitable Wittig inner salt to obtain a protected compound, and compound L22 is obtained after deprotection.

Wittig inner salt can be prepared by reacting a corresponding phosphonium with a strong base such as potassium bis(trimethylsilyl)amide (KHMDS) or sodium bis(trimethylsilyl)amide (NaHMDS), butyl lithium, sodium hydride or the like. Alternatively, Wittig inner salt can be prepared by other methods known in the art. Phosphonium can be prepared by reacting alkyl halide with triarylphosphine or trialkylphosphine (e.g. triphenylphosphine or tributyl phosphine), see Example 6.

In some embodiments of the present invention, phosphonium L23 (when L13 is L23, R8 in L13 is OP3) can be prepared according to synthetic route of reaction 8.

Synthetic route of reaction 8:

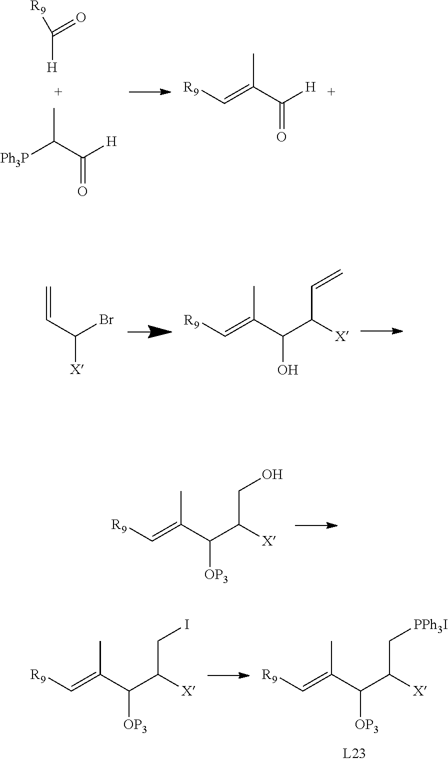

According to standard Wittig olefination (Meng, D., et. al. J. Org. Chem., 1996, 61: 7999)

1. Treating with organometallic reagent such as X′-allyl magnesium bromide.

2. Protecting the free hydroxy group by TES protecting group (OP3) using triethylsilyl chloride in DMF. Reacting with AD-mix-a by Sharpless, followed by oxidation of the cleaved bond using lead tetraacetate in ethyl acetate, and subjected to reduction using reducing agent such as sodium borohydride in methanol.

3. Reacting with I, imidazole and triphenylphosphine in toluene.

4. Reacting with triphenylphosphine in acetonitrile under reflux.

In some embodiments of the present invention, phosphonium L24 (when L13 is L24, R8 in L13 is NR15P) can be prepared according to synthetic route of reaction 9.

Synthetic route of reaction 9:

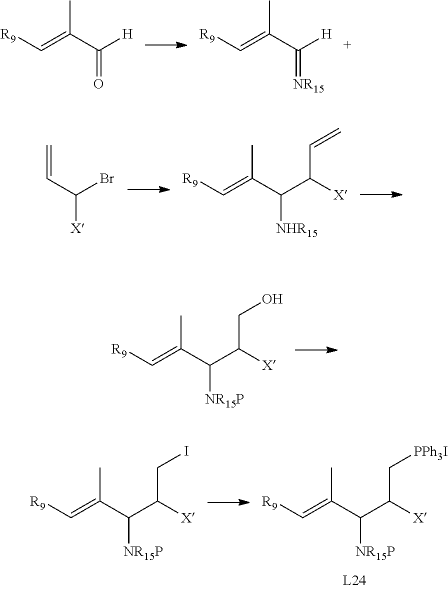

1. Prepared by treating with amine under anhydrous condition, e.g. using a catalytic amount of p-toluenesulfonic acid and subjected to azeotropy to remove water.

2. Treating with allylating agent such as 3-X′-allyl magnesium bromide.

3. The rest of the synthetic procedure is equivalent to the general procedure 3, 4, 5 described in synthetic route of reaction 8.

In some embodiments of the present invention, in the case R9 in L13 is thiazole or pyridine or the starting materials of synthetic route of reaction 9 are thiazole or pyridyl aldehyde compounds, thiazolyl aldehyde or pyridyl aldehyde can be prepared according to known method (Taylar, R. E. Tetrahedron Lett. 1997, 38:2061) or according to synthetic route of reaction 9-B, and then phosphonium compound, where R9 is thiazole or pyridine, can be prepared according to synthetic route of reaction 8 or 9.

Synthetic route of reaction 9-B:

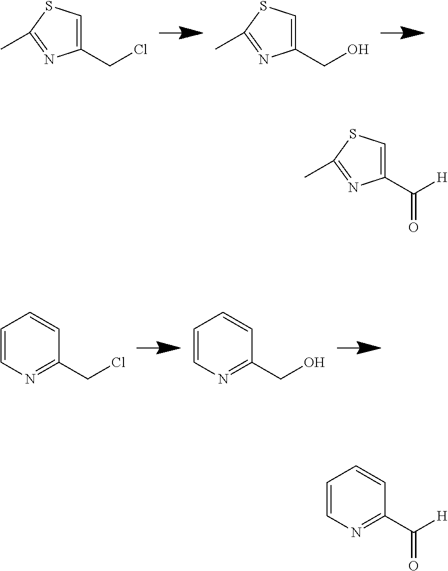

In some embodiments of the present invention, phosphonium L25 (when L17 is L25, X′in L17 is NR15P, R8 is methyl) can be prepared according to synthetic route of reaction 10.

Synthetic route of reaction 10:

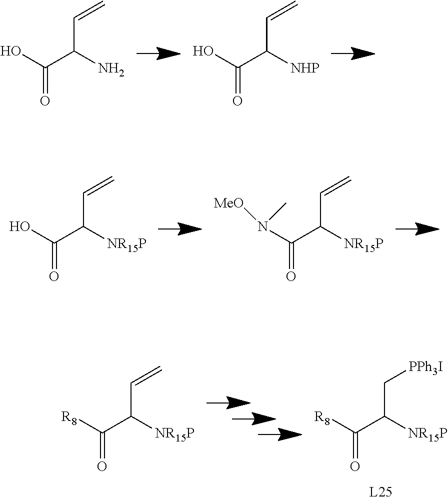

1. vinyl glycine is performed NH₂-protection, P is t-butyloxycarbonyl suitable for NH₂-protection (i.e., N-protecting).

2. When R15 is not H, compound is N-alkylated using haloalkane in the presence of base such as sodium hydroxide.

3. Treating with N.O-dimethylhydroxyamine and standard coupling agent such as EDCI and HOBT.

4. Treating with organometallic reagents such as alkyl or aryl magnesium halide or a hydroxamic ester.

5. The rest of the synthetic procedure is equivalent to the general procedure 3, 4, 5 described in synthetic route of reaction 8.

In some embodiments of the present invention, phosphonium L26 (when L17 is L26, X′in L17 is OP3, R8 is methyl) can be prepared according to synthetic route of reaction 11.

Synthetic route of reaction 11:

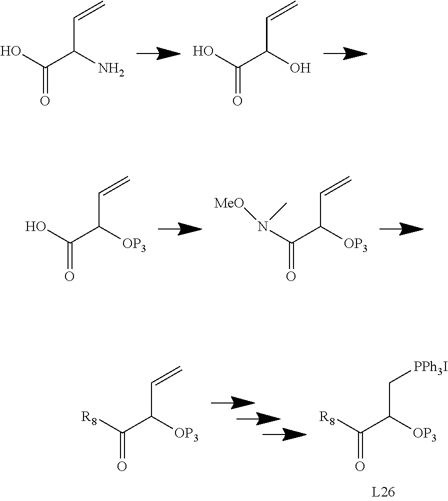

1. Treating vinyl glycine with nitric acid.

2. Protecting the free hydroxy group by TES protecting group (OP₃) using triethylsilyl chloride in DMF.

3. Treating with N.O-dimethylhydroxyamine and standard coupling agent such as EDCI and HOBT.

4. Treating with organometallic reagents such as alkyl or aryl magnesium halide or hydroxamic ester.

5. The rest of the synthetic procedure is equivalent to the general procedure 3, 4, 5 described in synthetic route of reaction 8.

Preferable compound of general formula I of the present invention and salts thereof can also be prepared by the total synthesis as shown in synthetic route of reaction 12 and 12B.

Synthetic route of reaction 12:

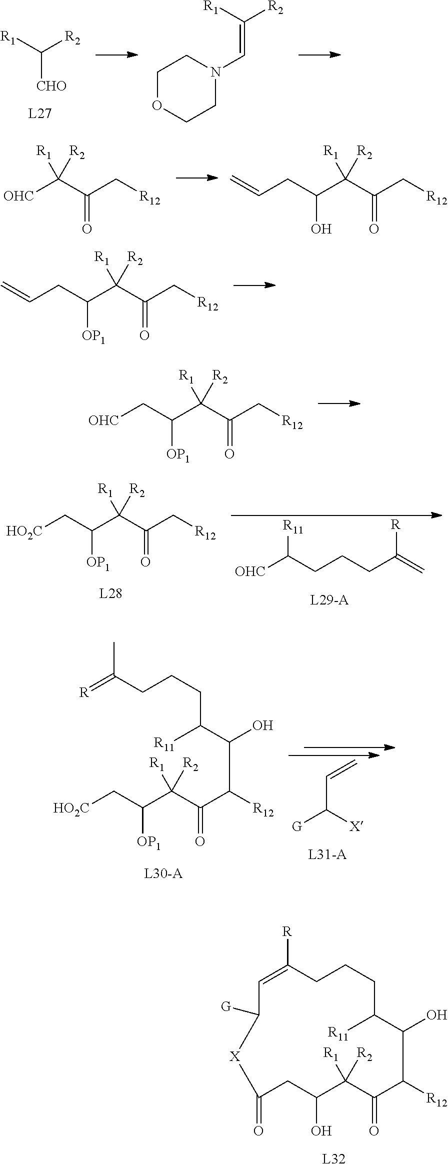

Synthetic route of reaction 12B

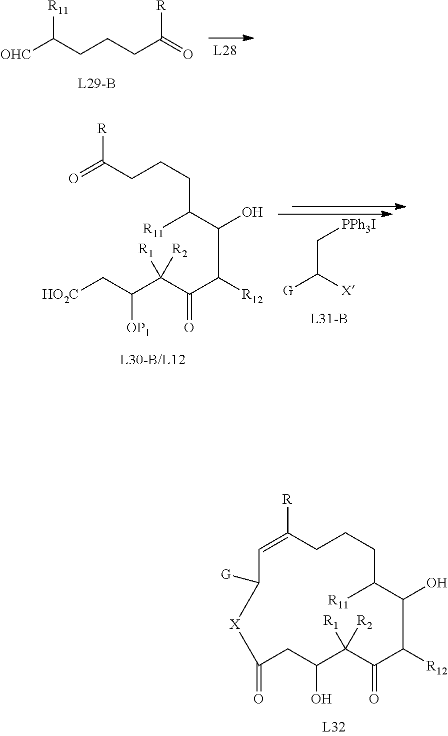

For example, compound L28, where P1 is an O-protecting group such as tertbutyldimethylsilyl, can be prepared from compound L27 according to known method (i.e. Nicolaou, K. C. et. at., Angew. Chem. Int. Ed. Engl. 1997, 36:166). Compound 29 can be prepared by know method (i.e., Schinzer, D. et. at., Eur. Chem. Chron. 1996, 1:7). Aldol reaction of compounds L28 and 29-A can afford compound L30-A. When X′in L31 is —OH, compound L30 and compound of 31 is coupled by using standard esterifying agent such as DCC and DMAP; or when X′in L31 is NHR15, compound L30-A and compound L31-A is coupled by using standard amide coupling agent such as DCC, BOP, EDC/HOBT, PyBroP; compound L32 can be prepared by olefin ring-closing metathesis using Grubbs' catalyst (RuCl₂(═CHPh)(PCY₃)₂; see Grubbs. et. al., Angew. Chem. Int. Ed. Engl. 1995, 34:2039) or Schrock's catalyst (see Schrock, R. R. et. al., J. Am. Chem. Soc. 1990, 112-3875) (Synthetic route of reaction 12).

Alternatively, compound L30-B (when R11, R12 is methyl, L30-B is L12) can be prepared by Aldol reaction of compound L28 and compound of 29-B. Compound L30-B and compound L31-B can be coupled appropriately by Wittig olefination, followed by macrocyclic lactonization or lactamization. When X′in L31 is —OH, compound L32 can be obtained by macrocyclic lactonization by reacting with a standard esterifying agent such as DCC and DMAP; or when X′in L31 is NHR15, compound L32 can be obtained by macrocyclic lactamization using standard amide coupling agent such as DCC, BOP, EDC/HOBT, PyBroP. See synthetic route of reaction 12B.

Synthetic route of reaction 13:

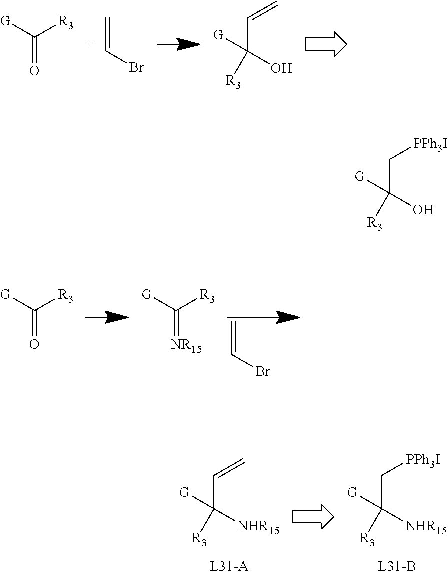

Compound L31 can be prepared from aldehyde compound following the synthetic reaction as shown in synthetic route of reaction 13. Aldehyde compound, where G is a substituted or unsubstituted alkyl, aryl, heteroaryl, dicyclic heteroaryl group or G is preferably as shown below, is vinylated by treating with a vinylating agent such as vinylmagnesium bromide, to afford compound L31-A where X′ is OH; when X′in L31-A is NHR15, the aldehyde compound where G is a substituted or unsubstituted alkyl, aryl group, heteroaryl group, dicyclic heteroaryl group or G is preferably as shown below, is reacted with amine under dehydration condition, followed by treating with a vinylating agent such as vinylmagnesium bromide, to afford compound L31-A where X′ is NHR15. Phosphonium L31-B can be prepared from compound L31-A according to the procedures equivalent to the general procedure 3, 4, 5 described in synthetic route of reaction 8.

G is preferably of the following formula:

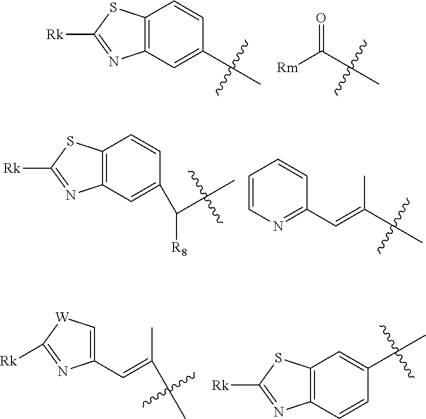

When G is a benzothiazole, benzothiazole aldehyde compound can be prepared according to the synthetic route of reaction 14 below, or aldehyde compound L33 can be prepared as described in example 8, and then compound L31 can be prepared following the synthetic route of reaction 13.

Synthetic route of reaction 14:

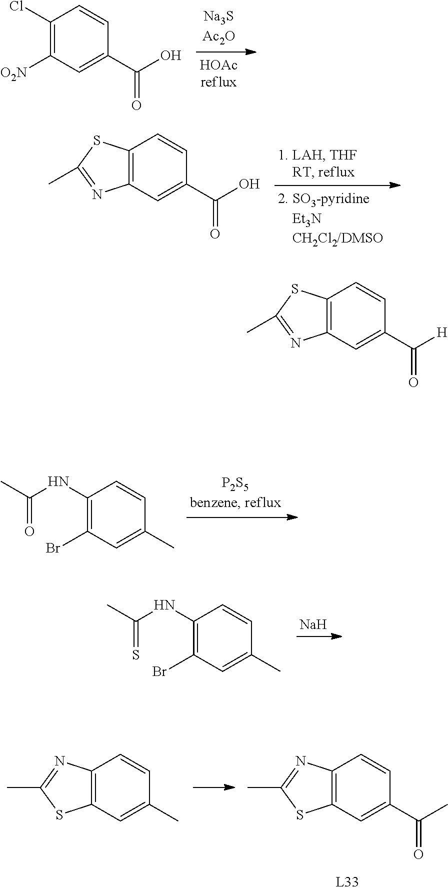

Compound of general formula I is preferably an epoxide (12,13-epoxy derivatives). Such compound can be prepared according to the epoxidation as shown in reaction 15 of the present invention described in Example 9.

Reaction 15:

Compound of general formula I is preferably a 12-hydroxylated derivative. Such compound as C-12 hydroxy compound of formula I can be prepared according to the general procedure for chemical modification described in reaction 16.

Reaction 16:

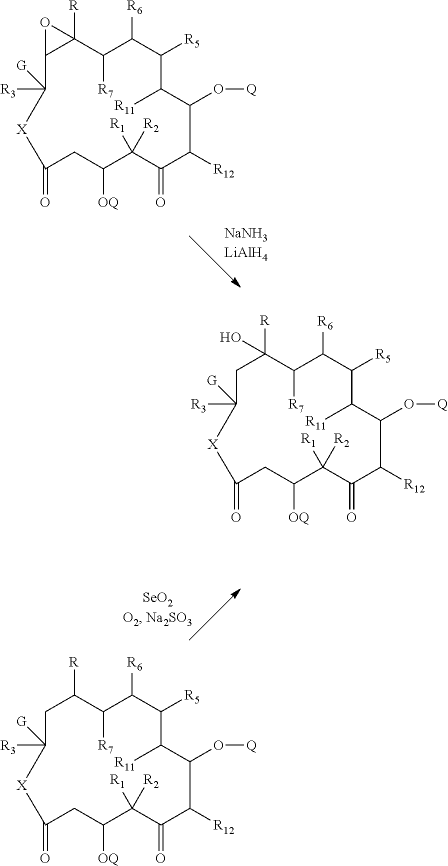

In other embodiments, the present invention provides a compound of general formula I having anyone of the following structures.

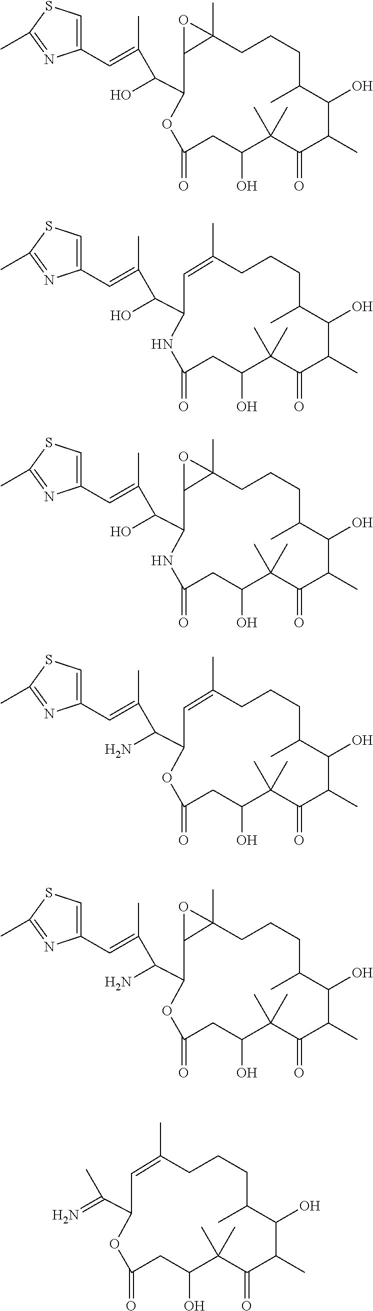

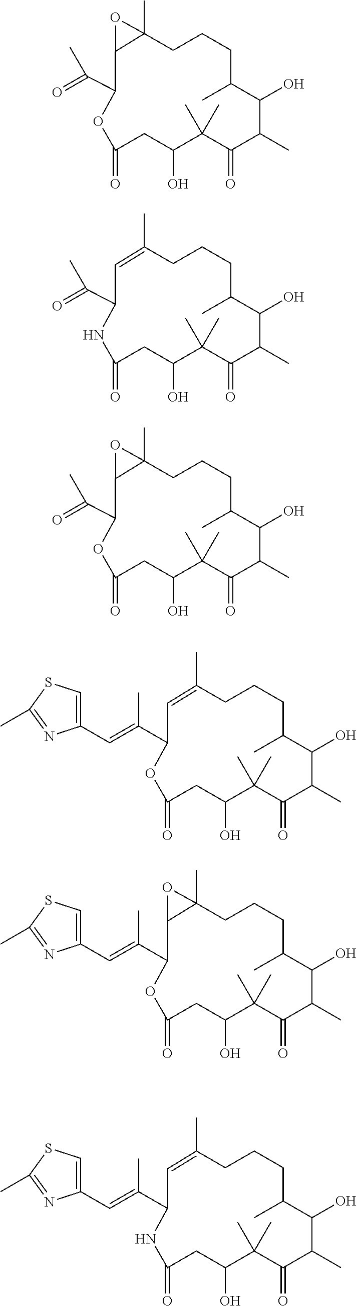

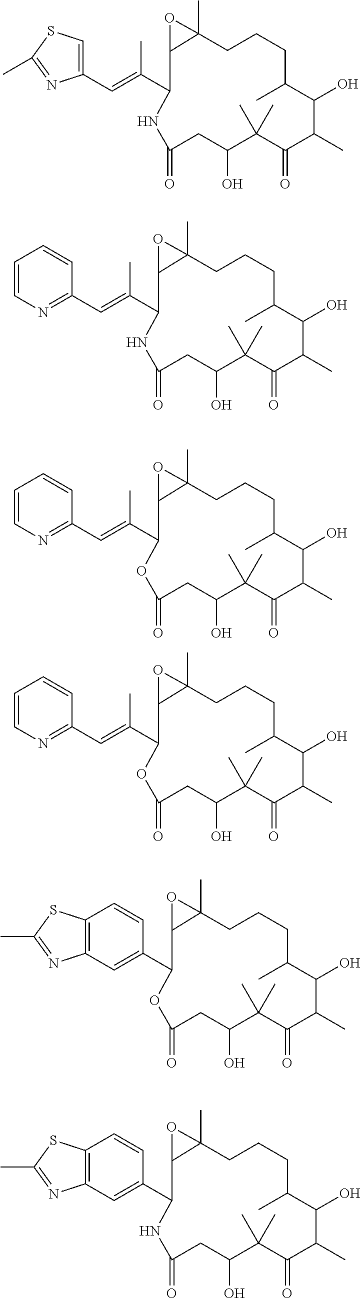

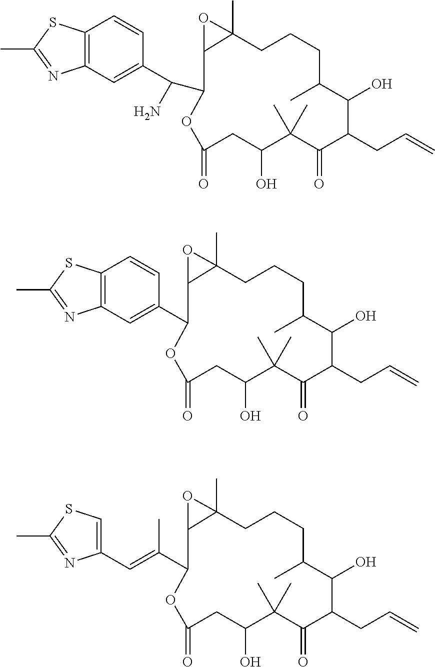

The compounds of the present invention can be screened by conventional assays known in the art. For example, cytotoxicity of the compound can be determined according to SRB assay described in Skehan et. al., J. Natl. Cancer Inst. 1990, 82: 1107, which is incorporated herein by reference.

The person skilled in the art is able to screen the compound of the present invention for microtubule polymerization using the conventional assays known in the art. For example, the compound for microtubule polymerization can be screened according to the method described in Gianakakou et. al., Intl. J. Cancer, 1998, 75: 63, which is incorporated herein by reference.

The present invention further provides a pharmaceutical composition, which comprises the compound of the present invention or a pharmaceutical acceptable salt, hydrate, polymorph, optical isomer, racemate, diastereomer or enantiomer thereof, and one or more conventional pharmaceutical carriers and/or diluents.

The pharmaceutical composition of the present invention further comprises one or more active agents in addition to the compound of the present invention or a pharmaceutical acceptable salt, hydrate, polymorph, optical isomer, racemate, diastereomer or enantiomer thereof.

The present invention further provides the use of the compound of the present invention in the preparation of a medicament in treating a proliferative disease. The compound of the present invention can be used in the preparation of a medicament for inhibiting excessive cell growth and termination of cell growth. Said proliferative disease is preferably selected from the group consisting of a tumor, multiple sclerosis, rheumatoid arthritis, atherosclerosis and restenosis.

The present invention provides a method for treating a proliferative disease using the compound of the present invention, said proliferative disease is preferably selected from the group consisting of a tumor, multiple sclerosis, rheumatoid arthritis, atherosclerosis and restenosis.

The present invention further provides a pharmaceutical composition for treating a proliferative disease, said proliferative disease is preferably selected from the group consisting of a tumor, multiple sclerosis, rheumatoid arthritis, atherosclerosis and restenosis.

The compound of the present invention can be of any forms, for example, a prodrug, a salt or ester of the compound of the present invention. The pharmaceutical composition may comprise at least one cyclodextrin and a acceptable carrier such as an alcohol (e.g., ethanol), ethylene glycol (propylene glycol), polyoxyethylene glycol (PEG), Tween, or Solutol etc. Said compound may be in any states, such as solid, semi-solid or liquid. The compound of the present invention can be formulated with a pharmaceutical acceptable carrier or diluent into a preparation for oral administration, intravenous administration or subcutaneous administration. Said pharmaceutical composition can be formulated according to standard methods employing solid or liquid carriers, diluents and additives suitable for the desired routes of administration. For oral administration, the compound of the present invention may be administered in the form of a tablet, capsule, granule, powder. The dosage range of the compound of the present invention is from about 0.05 to 200 mg/kg/day, which can be administered in a single dose or in a multiple dose of 2 to 5 portions.

The compound of the present invention can be formulated by other known method for preparing formulation containing drug of low solubility. For example, compounds can be formulated into emulsion with vitamin E and/or PEG polyacrylic acid derivatives (see WO00/71163 and U.S. Pat. No. 6,458,373 B1). Generally, the compound of the present invention is firstly dissolved in ethanol, followed by addition of vitamin E and/or PEG polyacrylic acid derivatives to form a therapeutic solution. Ethanol is removed, and a precursor emulsion is formed; or the precursor emulsion can be prepared by adding an aqueous solution comprising surfactants (stabilizers). For intravenous injection, the precursor emulsion can be dispersed to form a homogenous emulsion. For oral administration, the precursor emulsion is often placed in a gel capsule.

Based on the effects as an inhibitor of microtubule depolymerization, the action mechanism of the compound of formula I and that of anti-tumor agents such as paclitaxel and Epothilone is much the same, in which the function of cellular microtubule is disturbed mainly due to the induction of microtubule polymerization and microtubule stabilization, which results in the inhibition of cell division, cell migration, the intracellular signal transmission and protein secretion due to that all those actions depend on the rapid and effective dispolymerization of the microtubule. Therefore, the compound of formula I is effective in many proliferative diseases, such as solid tumors, liquid tumors (such as leukemia), etc.

The tumors treated by the compounds of the present invention include head and neck cancers; liver and gallbladder cancers; breast cancer, ovarian cancer, urogenital cancers, colorectal cancer, lung cancer, brain cancer, kidney cancer, leukemia, gastric carcinoma, liver cancer, glioma, malignant tumors and lymphoma. The method for treating said tumors or cancers comprises administering a therapeutically effective amount of the compound of the present invention to a cancer patient. The method can be repeated for preventing tumor migration or eradicating the tumors, if necessary. The use is especially due to the anti-angiogenesis activity of the compound of formula I. The compound of formula I can be used in possible combination of other therapeutic agents, especially one or more anti-proliferative, cell growth-inhibiting or cytotoxicity-suppressing compounds. In another aspect, the compound and composition of the present invention can be used in combination with other anti-tumor agents or therapies. In a further aspect, the compounds of the present invention can be used in the treatment of non-carcinoma diseases characterized as cellular hyper-proliferation. In yet another aspect, the compound of the present invention can be used in coating stent, like line-net tubes, for suspending cell growth and preventing restenosis or arterial re-blocking. In clinical trial, the compounds of the present invention may lead to one or more of the following phenomena: (i) increase of arterial flow; (ii) alleviation of clinical symptoms of the diseases; (iii) decrease of the rate of restenosis after heart valve surgery; or (iv) prevention/delay the progress of chronic atherosclerosis.

### EXAMPLES

### Example 1

### Bioconversion for 14-hydroxyl epothilone derivatives formation

A small freezing tube (1 ml) of Streptomyce sp. strain ATCC55098 was inoculated in 5 ml seed medium (20 g/L glucose, 20 g/L peptone, 10 g/L Yeast Extract, pH=7.0 adjusted with NaOH; used after sterilization), and then cultured in a shaking incubator at 30° C. for 2 days. Then, 2.5 ml culture was transferred to the fermentation medium (30 g/L baker's yeast; 15 g/L corn syrup, 1 g/L CaCO₃, 45 g/L cornstarch, 4, 23.8 g/L HEPES, 20 g/L dextrin, pH=7.0 adjusted with NaOH; used after sterilization), and cultured at 30° C. for 24 hours. 5-10 mg Epothilone D or appropriate compounds of the present invention was added to the culture medium, and further cultivated for 2-3 days. The conversion products of 14-hydroxylated Epothilone derivatives were isolated and recovered from the culture. For example, 14-hydroxyl epothilone D was isolated as the main bioconversion product from Epothilone D as the starting compound.

The MS (ESI+) of 14-hydroxyl epothilone D (C₂₇H₄₁NO₆S): 508 [M+H]⁺.

### Example 2

### Preparation of 15-Membered Thiazole Polyketide Lactone Compound HH1

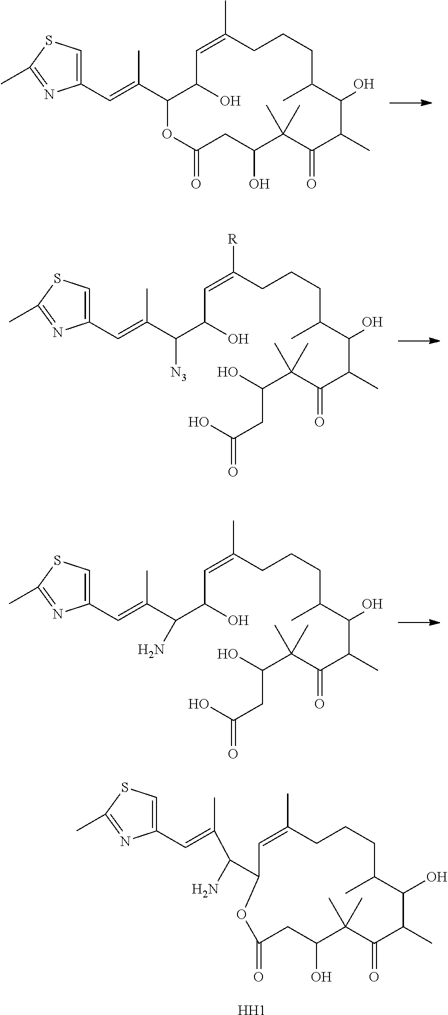

Step 1: A solution of 14-OH Epothilone D (2.62 g) in 55 ml degassed tetrahydrofuran (THF)/water (10:1 v/v) was treated with a catalytic amount of tetrakis(triphenylphosphine)palladium (0.58 g) under argon atmosphere, the suspension was stirred at 25° C. in argon for 30 minutes, and the resulting homogenous solution in light yellow color was then immediately treated with 25 ml of a degassed aqueous solution of sodium azide (0.49 g). Reaction was maintained at 45° C. for 1 hour, followed by diluting with 50 ml water, and extracting with ethyl acetate. The collected extracts were washed with saturated NaCl solution, dried over Na₂SO₄, followed by filtration and evaporation, and product was obtained after purified by SiO₂ chromatography.

Step 2a: a solution of the product (565 mg, 15-azide) obtained from step 1 above dissolved in 15 ml THF/water (10:1) was treated with a solution of trimethylphosphine (1.0M) in 3 ml toluene at ambient temperature under argon atmosphere for 2 hours. The mixture was concentrated, and product was obtained after purified by SiO₂ chromatography.

Step 2b: alternative method: a solution of the product (565 mg, 15-azide) obtained from step 1 above dissolved in 15 ml THF/water (10:1) was treated with triphenylphosphine (19 mg) under argon atmosphere for 2 hours. The mixture was concentrated, and the product was obtained after purified by SiO₂ chromatography.

Step 3: macrocyclic lactonization: At room temperature, tirethylamine and 2,4,6-trichlorobenzoyl chloride were added to the THF solution of the product obtained in step 2. After 20 minutes, the mixture was diluted with toluene, and added dropwise to a warm solution of 4-(dimethylamino)pyridine in toluene within 4 hours. After addition, the mixture was concentrated, and purified by SiO₂ chromatography.

MS (ESI+) of 15-membered thiazole polyketide lactone compound HH1 (C₂₇H₄₂N₂O₅S): 507 [M+H]⁺.

### Example 3

### Preparation of 15-Membered Thiazole Polyketide Lactam Compound HH2

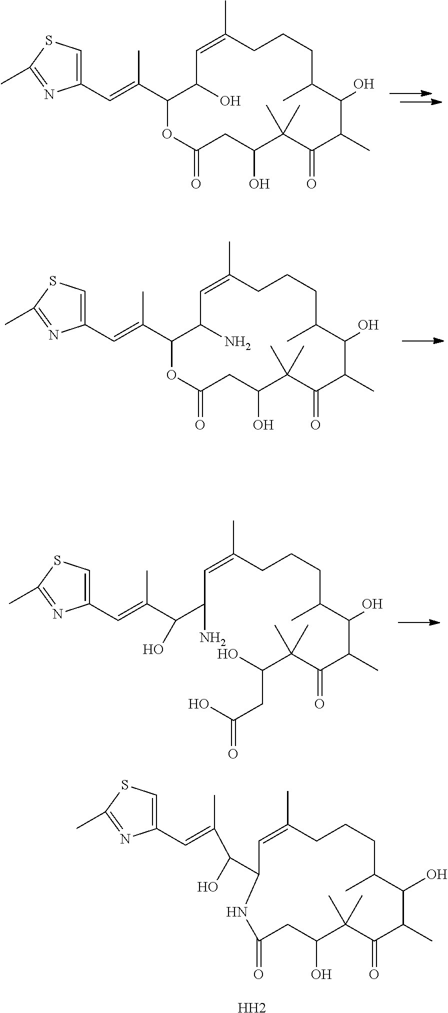

1. A solution of 3,7-O-(tert-butyldimethylsilyl)- 14-OH Epothilone D (30 mg) in 1.5 ml anhydrous THF was cooled to 0° C. After being treated with diphenylphosphoryl azide (15.5 μl) for 5 minutes, 8.8 μl of 1,8-diazabicyclo(5.4.0)undec-7-ene (DBU) was added, and the reaction mixture was stirred at 0° C. for 2 hours, and was then warmed to room temperature and stirred for 20 hours. Subsequently, the solution was added with 30 ml ethyl acetate, and washed with water (2×10 ml), the collected aqueous phase was then extracted with ethyl acetate (2×15 ml), dried over Na₂SO₄, filtered and evaporated. 3,7-O-((tert-butyldimethylsilyl) protected 14-azide Epothilone was then purified by SiO₂ chromatography.

2a. A solution of azide (14 mg) (obtained from the above step) in 0.3 ml THF and trimethylphosphine (33 μl 1M THF solution) was stirred for 5 minutes, followed by treating with 80 μl of water, and stirring for 3 hours. At this time, azide was thoroughly consumed, phosphoryl imine was completely converted to amine upon addition of 50 μl of a 28% NH₄OH aqueous solution. After being stirred at 25° C. (room temperature) for 1 hour, solvent in the mixture was evaporated under vacuum. Chromatographic purification was performed with silicon gel (10% methanol in chloroform solution) to afford 3,7-O-(tert-butyldimethylsilyl) protected 14-amino Epothilone.

2b. alternative method: 18 mg of Lindlar catalyst was suspended and saturated in 0.5 ml ethanol, followed by addition of the azide (obtained from the above step) dissolved in ethanol-methanol mixture, stirred at room temperature for 30 minutes, the resulting suspension was filtered through celite, washed with ethyl acetate, dried under vacuum.

3. At room temperature, a methanol solution of the product obtained in step 2 above was treated with 1N NaOH, the reaction was monitored with TLC or HPLC, and reaction was stopped by adding phosphate buffer of pH4, methanol was then removed by vacuum evaporation, aqueous residue was then extracted with ethyl acetate, dried over Na₂SO₄, filtered and evaporated.

4a. the product obtained in step 3 above (540 mg) was dissolved in 15 ml acetonitrile/dimethylformamide (20:1 v/v) solution, which was then cooled to 0° C., and treated with 1-hydroxybenzotriazole (0.135 g) and 1-(3-dimethylaminopropyl)-3-ethylcarbodiimide hydrochloride (0.5 g). The resulting mixture was then heated to ambient temperature and kept for 12 hours, followed by diluting with water and extracting with ethyl acetate, the extracts were then sequentially washed with water, saturated NaHCO₃, and saturated NaCl solution, dried over Na₂SO₄, filtered and evaporated. The protected 15-membered thiazole polyketide lactam compound was obtained after purified by SiO₂ chromatography. The resulting protected product was then subjected to deprotection by dissolving in 1:1 trifluoroacetic acid and dichloromethane to obtain the end product.

4b. alternative method: Under Ar atmosphere, a solution of compound obtained in step 2 above (0.33 g) in 250 ml degassed DMF was treated with solid (0.42 g) and diphenylphosphoryl azide (0.54 ml) at 0° C., the resulting suspension was stirred at 4° C. for 24 hours, followed by diluting with phosphate buffer (250 ml, pH7.0) at 0° C., and extracting with ethyl acetate (4×100 ml), the organic phase was washed with 10% aqueous solution of lithium chloride (2×100 ml), and then dried over Na₂SO₄, filtered and evaporated. The product was obtained after purified by SiO₂ chromatography.

MS (ESI+) of 15-membered thiazole polyketide lactam compound HH2 (C₂₇H₄₂N₂O₅S): 507 [M+H]⁺.

### Example 4

### Preparation of 15-Membered Thiazole Polyketide Compound HH3

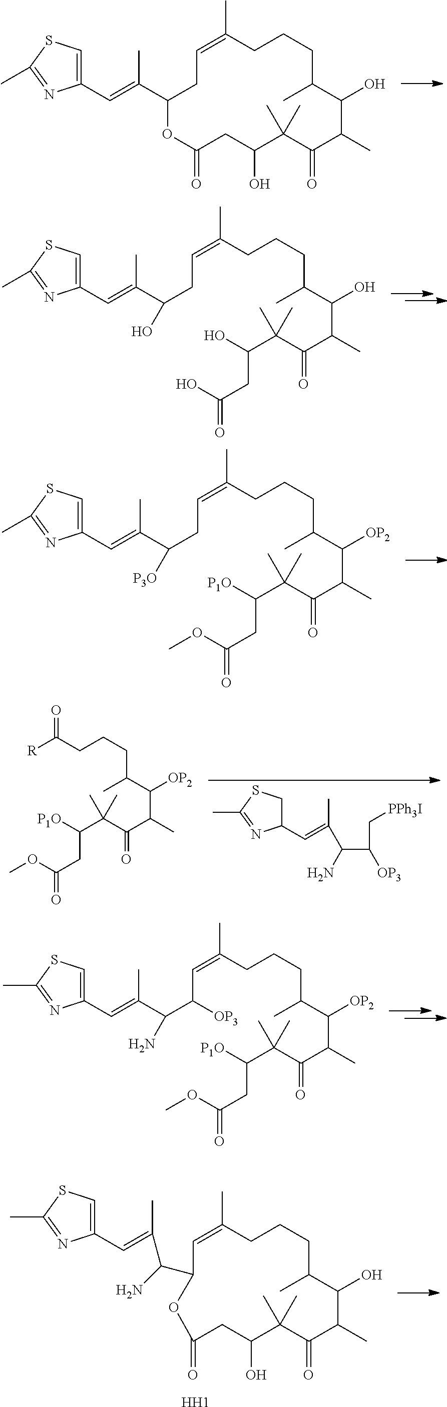

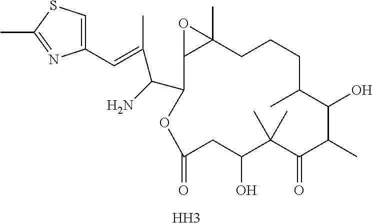

1. Ring opening: Epothilone D (8.4 mg) was dissolved in 125 μl of DMSO, and diluted with 5 ml phosphate buffer (pH7.0), followed by addition of 200 units of Pig liver esterase for overnight hydrolysis at 37° C. pH of the mixture was adjusted to pH=4.5 with 1N HCl, and then extracted with dichloromethane (2×5 ml), the extracts were dried over Na₂SO₄, filtered and evaporated. The product was obtained by SiO₂ chromatography (eluted with ethyl acetate containing 1% acetic acid). MS (ESI+): 510 [M+H]⁺.

2. Carboxymethylation: product (1 mg) from step 1 above was dissolved in 0.5 ml mixture of 2:7 methanol:toluene, followed by addition of 2 drops of trimethylsilyl diazomethane, after 10 minutes at 25° C., mixture was evaporated, and purified by SiO₂ chromatography to obtain the pure product. MS (ESI+): 524 [M+H]⁺.

3. Protection for free hydroxy group: the product obtained in step 2 above (20.4 mg) was dissolved in 2 ml anhydrous dichloromethane, followed by addition of 2,6-dimethylpyridine (2,6-lutidine) (23 μl), cooled to −14° C., added dropwise with t-butyldimethysilyl triflate (32 μl), Reaction proceeded for 30 minutes, followed by addition of 2,6-dimethylpyridine (2,6-lutidine) (33 μl) and t-butyldimethysilyl triflate (65 μl), after reacting for 12 hours, saturated NaHCO₃ (5 ml) was added, which was then extracted with dichloromethane (2×5 ml), the resulting extracts were then dried over Na₂SO₄, filtered and evaporated. The product (tri-3,7,15-TBS-Epo D) was then purified by SiO₂ chromatography. MS (ESI+): 852 [M+H-CH₃]⁺.

4. Cleavage of molecule: product (6.4 mg) from step 3 above was dissolved in 2 ml anhydrous dichloromethane, and then cooled to −78° C. Ozone was bubbled through the solution for about 2 minutes, and the solution became light blue in color. The solution was then added with triphenylphosphine (8 mg) and warmed to room temperature within 30 minutes. The mixture was evaporated, and purified by SiO2 chromatography to afford the pure product (L12 in the case R, R1, R2 is methyl). MS (ESI+): 573 [M+H]⁺.

5. Preparation of phosphonium: small molecule compound L24 (wherein R9 is thiazole, X′═OP3, R15=H) can be prepared according to the general procedure of synthetic route of reaction 9.

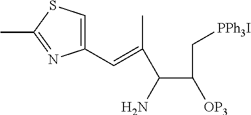

6. Wittig: product (18 mg) from step 5 above was dissolved in 0.5 ml of anhydrous tetrahydrofuran, and cooled to 0° C., followed by addition of sodium bis(trimethylsilyl)amide (31 μl), the solution became brown in color, the mixture was cooled to −20 C, and added with 7.3 mg of product obtained in step 4 above dissolved in 0.5 ml of anhydrous THF. After reacting for 10 minutes, saturated NaHCO₃ (4 ml) was added, which was then extracted with dichloromethane (2×2 ml), the extracts were then dried over Na₂SO₄, filtered and evaporated. The product was purified by SiO₂ chromatography. MS (ESI+): 867 [M+H-CH₃]⁺.

7. Deprotection of methyl ester: product (2.2 mg) from step 6 above was dissolved in 0.5 ml t-butyl alcohol/water (2:1), and treated with 1M LiOH (40 μl), reaction was stirred at room temperature for 48 hours. The product was purified by SiO₂ chromatography.

8. Deprotection of 14-OH (removal of P3): product from step 7 above was dissolved in a mixture of acetonitrile, water and acetic acid. Reaction was monitored for the disappearance of the starting material by TLC or HPLC, and was dried by vacuum evaporation. Or product from step 7 was subjected to hydrolysis using desilylating agent or acid in inert solvent or mixture thereof such as TASF or HF-pyridine in THF to achieve selective desilylation.

9. Macrocyclic lactonization: 87 μl of triethylamine and 68 μl of 2,4,6-trichlorobenzoyl chloride (Aldrich) were added to a solution of 0.216 g of hydroxy acid product from step 8 in 3 ml THF at room temperature. The resulting solution was stirred at 0° C. for 1 hour, which was then added dropwise in 4 hours to a warm solution of 0.354 g N,N-(dimethylamino)-pyridine in toluene at room temperature. After addition, the solution was stirred for 2 hours, and concentrated by evaporation. The product was obtained after purified by SiO₂ chromatography.

10. Deprotection: the protected product (67 mg) was dissolved in 1.5 ml THF, and treated with hydrogen fluoride-pyridine (0.6 ml) at 0° C. After 20 minutes, reaction was warmed to room temperature, and kept at this temperature for 5 hours, and then cooled to 0° C. again. Methoxytrimethylsilane (6 ml) was slowly added, the resulting mixture was warmed to room temperature, and then subjected to evaporation to afford an oily product, which was then purified by SiO₂ chromatography and a pure product was thus obtained. MS (ESI+) of 15-membered thiazole polyketide lactam compound (C₂₇H₄₂N₂O₅S): 507 [M+H]⁺.

11. Epoxidation: product from step 10 above was subjected to epoxidation according to Example 9 to obtain the 15-membered epoxy thiazole polyketide compound HH3 (C₂₇H₄₂N₂O₆S). MS (ESI+): 523 [M+H]⁺.

### Example 5

### Preparation of 15-Membered Thiazole Polyketide Lactam Compound HH4

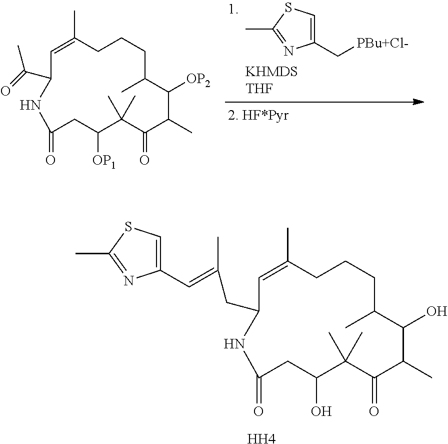

At −30° C., to a solution of 2-methylthizole-4-methyl tri-n-butyl chloride (0.64 g) in 3 ml THF was added dropwise a solution of 0.5M potassium bis(trimethylsilyl)amide (KHMDS) in toluene (1.5 ml) in 10 minutes. The resulting solution was warmed to 0° C. in 40 minutes, and cooled to −70° C., followed by dropwise addition of a solution of formula L20 (where Rm was methyl, X was NH in L20) ketone compound (85 mg) in 1 ml THF (A. Rivkin et. al., J. Am. Chem. Soc. 2003, 125:2899). After 20 minutes, the resulting mixture was warmed to −30° C. in 1 hour, and kept at this temperature for 2 hours. Reaction was stopped upon addition of a saturated aqueous solution of ammonium chloride, which was then extracted with ethyl acetate, the extracts were then washed with brine water, dried over MgSO₄, filtered and evaporated. The protected product was obtained after purified by SiO₂ chromatography.

The thus protected product (67 mg) was dissolved in 1.5 ml THF, and treated with hydrogen fluoride-pyridine (0.6 ml) at 0° C. After 20 minutes, reaction was warmed to room temperature, and kept at this temperature for 3.5 hours, and then cooled to 0° C. again. Methoxytrimethylsilane (6 ml) was slowly added, the resulting mixture was warmed to room temperature, and then subjected to evaporation to afford an oily product, which was then purified by SiO₂ chromatography and a pure product was thus obtained. MS (ESI+) of 15-membered thiazole polyketide lactam compound HH4 (C₂₆H₄₀N₂O₄S): 477 [M+H]⁺.

In the case L20 ketone compound, where Rm was methyl, X was 0, was used in the reaction, 15-membered thiazole polyketide lactam compound HH5 (C₂₆H₃₉NO₅S) was obtained. MS (ESI+): 478 [M+H]⁺.

15-membered thiazole polyketide lactam compound HH5 was subjected to epoxidation as shown in reaction 15 of the present invention as described in Example 9 to obtain 14-epoxy thiazole polyketide compound HH6 (C₂₆H₃₉NO₆S). MS (ESI+): 494 [M+H]⁺.

### Example 6

### Preparation of (2-methyl-4-thiazole)methyl tributylphosphonium chloride

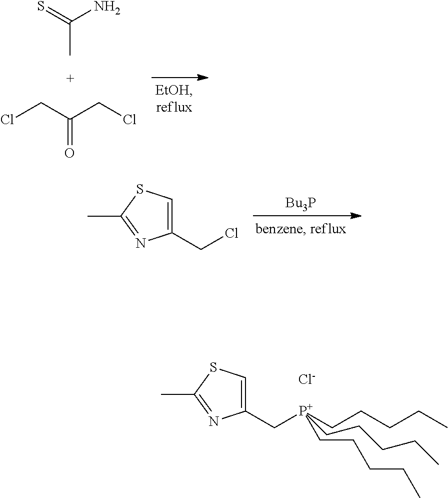

A mixture of 1.3-dichloroacetone (30 mmol) and thioacetamide (30 mmol) was dissolved in 21 ml anhydrous ethanol, and reflux overnight under nitrogen. Solution was concentrated under vacuum, residue was dissolved in water (100 ml), pH was adjusted to pH 8.0, followed by extraction with diethyl ether. Sequentially added with saturated NaHCO₃ (4 ml), extracted with dichloromethane (2×2 ml), the collected extracts were washed with saturated NaHCO₃, water, and brine water, the organic phase was dried over Na₂SO₄, filtered and evaporated. The product, 2-methyl-4-chloromethylthiazole, was obtained after purified by SiO₂ chromatography.

To a solution of 2-methyl-4-chloromethylthiazole (557 mg) in 3 ml benzene was added dropwisely tri-n-butylphosphine. The resulting solution was reflux overnight under nitrogen, solution was concentrated under vacuum, and residue was subjected to crystallization by adding a mixture of 1:1 (v/v) diethyl ether and hexane. Solid was filtered and washed with a small amount of hexane, and then dried to afford the phosphonium salt as a white solid.

### Example 7

### Preparation of 15-Membered Pyridyl Polyketide Lactam Compound HH9

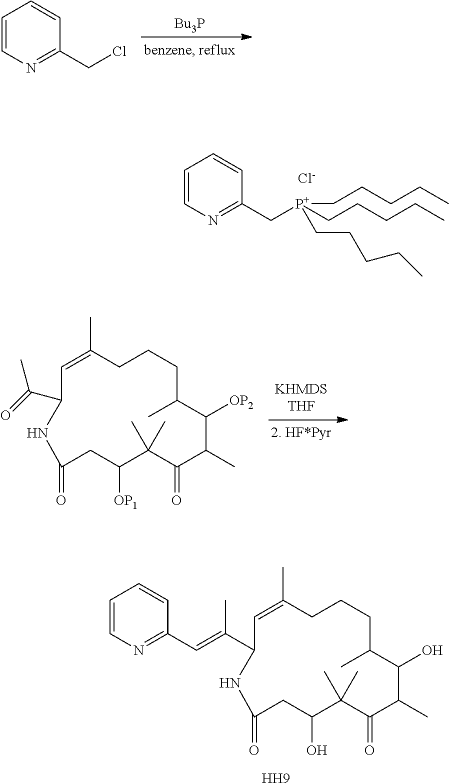

Preparation of (2-pyridyl)methyl tri-n-butylphosphonium chloride. To a solution of 2-(chloromethyl)pyridine (10 mmol) in 15 ml benzene was added dropwisely tri-n-butylphosphine (10 mmol) under nitrogen, the resulting solution was reflux for 18 hours, and then cooled to room temperature. The solution was concentrated under vacuum. Air was excluded by all means. To the residue was added diethyl ether and a white solid was obtained. Filtrate was filtered out. The thus obtained white solid was dried with diethyl ether under vacuum in nitrogen atmosphere, and product was obtained in the form of a white powder.

Compound was prepared according to the procedure of Example 5, (2-pyridyl)methyl tri-n-butylphosphonium chloride was reacted with formula L20 ketone (in the case X was NH). The solution was warmed to −10° C. before the end of the reaction. A pure product, 15-membered pyridyl lactam compound HH9 (C₂₇H₄₀N₂O₄) was obtained. MS (ESI+): 457 [M+H]⁺.

### Example 8

### Preparation of benzothiazolephosphonium compound L34 and 14-epoxybenzo thiazole polyketide compound HH8

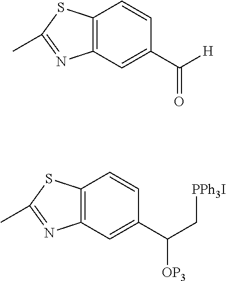

To a solution of dimethylbenzothiazole (Fluka, Buchs) (8 g) in 50 ml tetrachloromethane was added N-bromosuccinimide (10.5 g), the solution was radiated with a tungsten lamp and heated for 4 hours to 80° C. The resulting mixture was cooled to room temperature, and then filtered, solvent was evaporated. The thus obtained oily matter was mixed with an aqueous solution of acetic acid (50%, 100 ml) and hexamethylenetetramine (23.4 g), the mixture was heated for 2 hours to 110° C. Reaction was then stopped by adding water, and extracted with ethyl acetate. The collected extracts were sequentially extracted with saturated NaHCO₃, water, and brine water, dried over Na₂SO₄, filtered and evaporated. The product was obtained after purified by SiO₂ chromatography.

Dimethylbenzothiazolephosphonium L34 can be prepared according to procedure 2, 3, 4, 5 of synthetic route of reaction 8, i.e. reacting dimethylbenzothiazole aldehyde with vinylmagnesium bromide, P3 protection, Sharpless Reduction, iodization and finally reacting dimethylbenzothiazole iodide with triphenylphosphine.

Benzothiazolephosphonium L34 prepared in the present Example was reacted with compound L12 (compound L12, where R, R1, R2 was methyl) according to the preparation procedure of Example 4 (step 6-11) of the present invention, 14-epoxybenzo thiazole polyketide compound HH8 (C₂₇H₃₉NO₆S) was obtained. MS (ESI+): 504 [M+H]⁺.

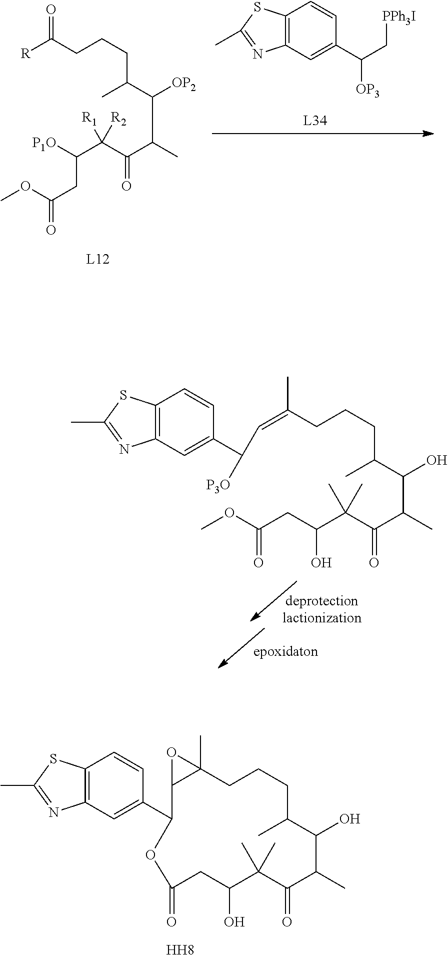

### Example 9

### Epoxidation

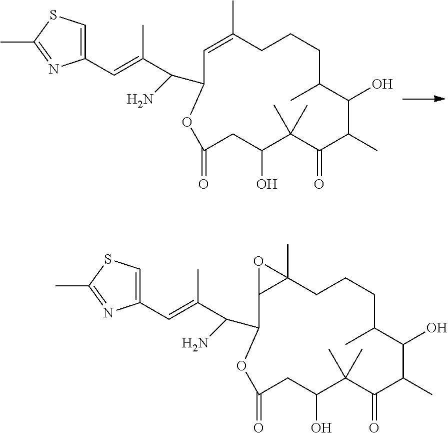

The present example describes the epoxidation reaction of compound of formula I as A-Q links together to form a C═C bond. At −78° C., to a solution of deoxygenated compound (505 mg) of the present invention in 10 ml CH₂Cl₂ was added dropwise a solution of dimethyldiethylene oxide (0.1M in acetone, 17 ml). The resulting mixture was heated to −50° C., and kept at this temperature for 1 hour, followed by addition of another portion of the dimethyldiethylene oxide (5 ml), the reaction proceeded for 1.5 hours at −50° C. Reactants were dried at −50° C. under nitrogen atmosphere. The product was obtained after purified by SiO₂ chromatography.

This general procedure may be suitably applied to other compounds of the present invention for preparing the corresponding 12,13-epoxy derivatives.

### Example 10

### Biological Activity Assay

In the study, sulforhodamine B (SRB) was used for screening the selective compounds of the present invention according to their anti-tumor activity on four different tumor cell lines. In SRB assay, the cultured cells were trypsinized, then counted and diluted to a suitable concentration (5000-7500 cells/100 μl) with a culture medium. The cells were inoculated into a 96-well microtiter plate at 100 μl suspension/well. The test compounds were diluted in the culture medium to 2×1000 nM˜2×0.001 nM, and added to each well in 100 μl after 20 hours. The cells were then cultured for 3 days, fixed with 100 μl 10% trichloroacetic acid at 4° C. for 1 hour, and then stained with 0.2% SRB/1% acetic acid at room temperature for 20 min. The unbound dye was rinsed with 1% acetic acid. The bound dye was dissolved with 200 μl 10 mM Tris-base. The amount of the bound dye was calculated from OD value detected at wavelength of 515 nm. The amount of the bound dye was in direct proportion to the total amount of cell proteins. The data was analyzed with Kaleida Graph Program, and the half inhibitory concentration (IC50) was calculated. Epothilones D and B were detected in parallel for comparison. The results of the cytotoxic experiments on the tested selective compounds according to the present invention are shown as follows. The other compounds according to the present invention may also be detected with the similar method.

The action mechanism was detected by cell-based microtubule polymerization assay. In the assay, MCF-7 cells cultured in a 35 mm dish were treated with 1 μM of the compound according to the present invention at 37° C. for 1 hour. The cells were washed twice with 2 mL PBS without Ca and Mg, and then were treated with 300 μL, lysis solution (20 mM Tris, pH 6.8, 1 mM MgCl₂, 2 mM EDTA, 1% Triton X-100, with proteinase inhibitor) for 5-10 min to lyse the cells. The lysate was removed to a 1.5-mL Eppendorf tube, and centrifuged at 18000 g for 12 min at room temperature. The supernatant containing soluble or unassembled microtubule was separated from the granular precipitate containing insoluble or assembled microtubule and removed to a new tube. The granular precipitate was re-suspended with 300 μL, lysis solution. Each sample was analyzed by SDS-PAGE and immunoblotting using anti-β-microtubule antigen (Sigma). The amount of β-microtubule on the blotting was analyzed with NIHImage program for detecting the changes of microtubule polymerization in the cells.

The microtubule polymerization assay demonstrated that 15-membered thiazole derivatives had the same action mechanism as Epothilones, and exhibited similar dynamics and effects under the study condition. The other compounds according to the present invention may also be detected with the same method.

| | Activity against cancer cell lines(IC₅₀, nM) | | | |
|---|---|---|---|---|
| | MCF-7 | NCI/ADR-RES | | NCI-H460 |
| | (Breast | (MDR Breast | SF-268 | (Lung |
| Compounds | Cancer) | Cancer) | (Glioma) | Cancer) |
| Epo D | 13 | 42 | 18 | 17 |
| Epo B | 0.5 | 5 | 0.8 | 0.7 |
| 15-aminoepoxy- | 1 | 3 | 1 | 0.5 |
| thiazole polyketide | | | | |
| HH3 | | | | |
| 14-epoxythiazole | 0.2 | 1 | 1 | 0.5 |
| polyketide HH6 | | | | |
| 14-epoxybenzothiazole | 0.5 | 0.5 | 1 | 0.2 |
| polyketide HH8 | | | | |

1. A 15-membered thiazole polyketide compound of the following general formula I: 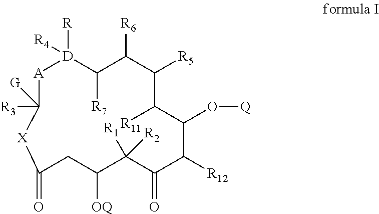
wherein,
as A-D represents a C═C bond of formula (a) or an epoxy group of formula (b), R₄ is not exist, 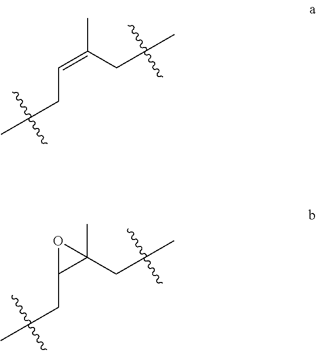
as A-D represents a C—C bond, R₄ represents a hydroxy group or H,
G is selected from a substituted or unsubstituted alkyl group, a substituted or unsubstituted aryl group, a heteroaryl group, a heterocyclic group, a cycloalkyl group, or any one selected from the following formulae: 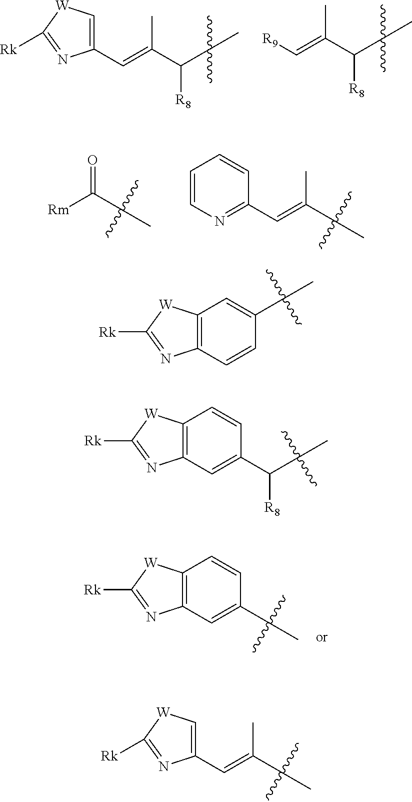
Q is selected from H, a C₁₋₄ alkyl group, NH₂ or a hydroxy-protecting group;
R₁, R₂ are each independently selected from H or a substituted or unsubstituted C₁₋₄ alkyl group, or together form a cycloalkyl group;
R₈ is selected from H, a hydroxy group, a substituted or unsubstituted C₁₋₈ alkyl group or NH₂, N₃ or NR₁₃R₁₄;
X represents O, S or N—R₁₅, wherein R₁₅ represents H, NR₁₆R₁₇, a substituted or unsubstituted C₁₋₄alkyl group, a substituted or unsubstituted aryl group, a cycloalkyl group or a heterocyclic group;
R₉ is selected from H, a substituted or unsubstituted C₁₋₄ alkyl, an aryl group, a heteroaryl group, a cycloalkyl group or a heterocyclic group;
R₁₂ is selected from H, an allyl group, a hydroxy group, NH₂ or a substituted or unsubstituted C₁₋₆ alkyl;
Rm is selected from H, methyl, NR₁₆R₁₇ or halomethyl;
Rk is selected from H, a substituted or unsubstituted C₁₋₄ alkyl group, an aminoalkyl group, a hydroxyalkyl group or a haloalkyl;
R₃, R₄, R₅, R₆, R₇, R₁₁, R₁₃, R₁₄, R₁₆, R₁₇ are each independently selected from H, a hydroxy group, NH₂ or a substituted or unsubstituted C₁₋₆ alkyl group, wherein R₅, R₆ may together form a C═C bond;
R is selected from H, trifluoromethyl, a substituted or unsubstituted alkyl group or halogen;
W represents S or O, NH, N-alkyl;
or a pharmaceutical acceptable salt, hydrate, polymorph, optical isomer, racemate, diastereomer or enantiomer thereof.

2. The compound according to claim 1, wherein G in said compound is selected from:

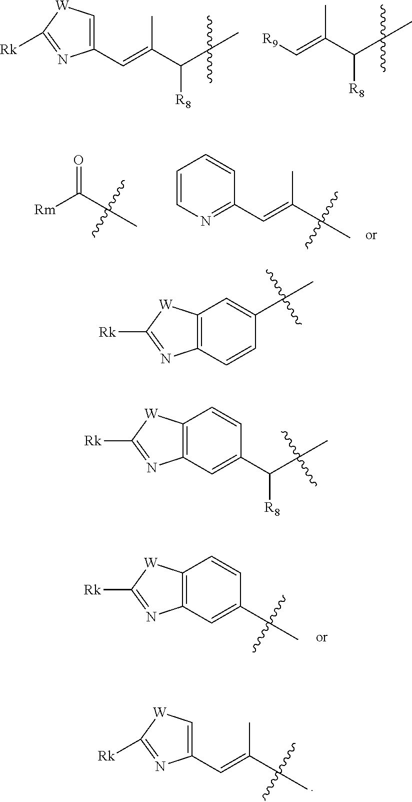

3. The compound according to claim 1, wherein said compound is represented by the structure of the following general formula II: 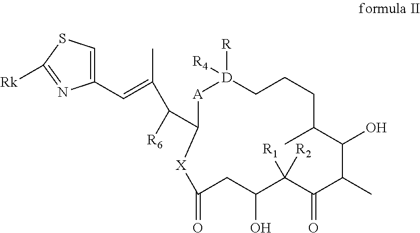
wherein, X is NR₁₅ or O;
R₈ is NHR₁₅ or OQ;
each of the other groups has the same meaning as defined in claim 1.

4. The compound according to claim 1, wherein said compound is represented by the structure of the following general formula III: 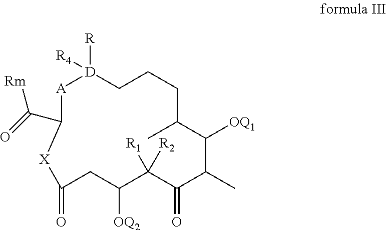
wherein, Q₁ and Q₂ each independently represents H, a C₁₋₄alkyl group, NH₂ or a hydroxy-protecting group; each of the other groups has the same meaning as defined in claim 1.

5. The compound according to claim 1, wherein said compound is represented by the structure of the following general formula IV: 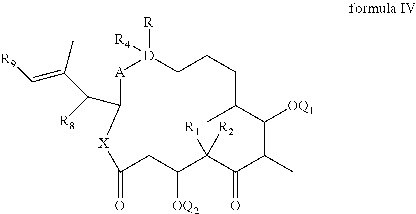
wherein, each group has the same meaning as defined in claim 1.

6. The compound according to claim 1, wherein said compound is represented by the structure of the following general formula V: 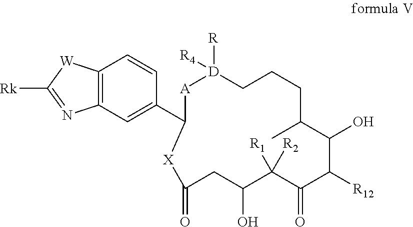
wherein, X is NR₁₅ or O; R₁₅ is H, a methoxy group or an alkyl group; R₁₂ is H, an allyl group, a substituted or unsubstituted C₁₋₆ alkyl group.

7. The compound according to claim 1, wherein said compound is selected from the following compounds:

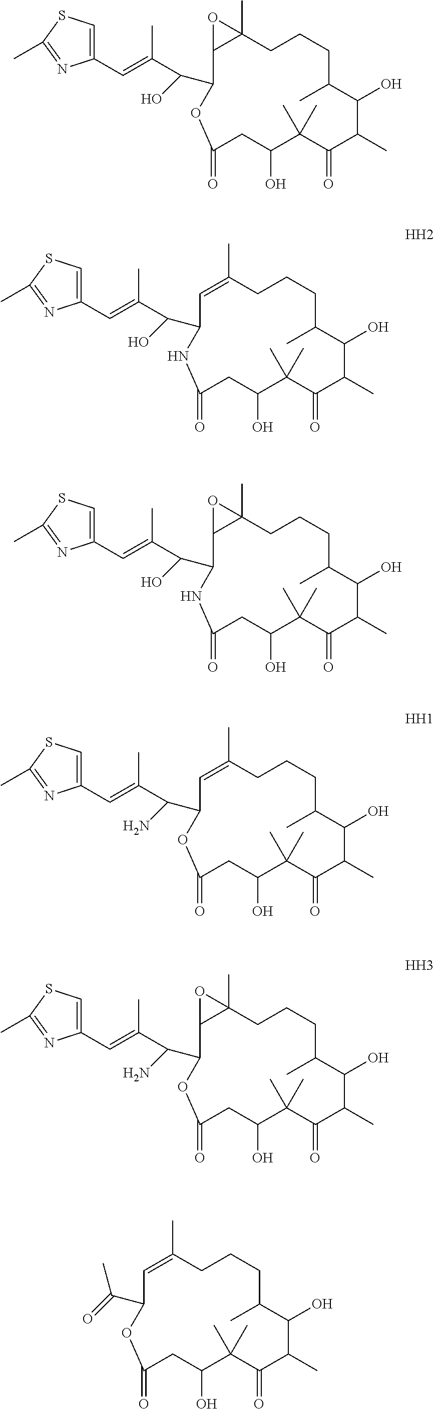

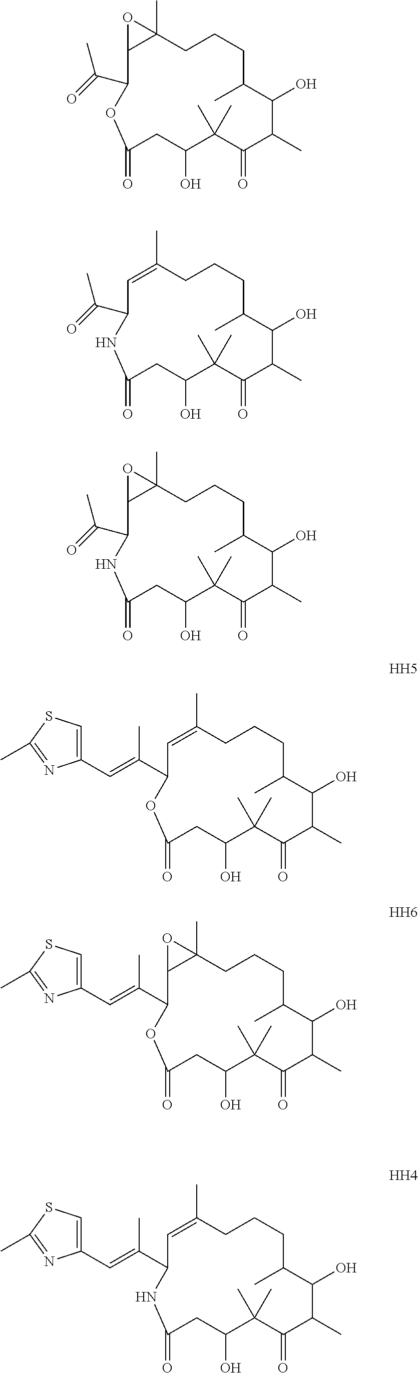

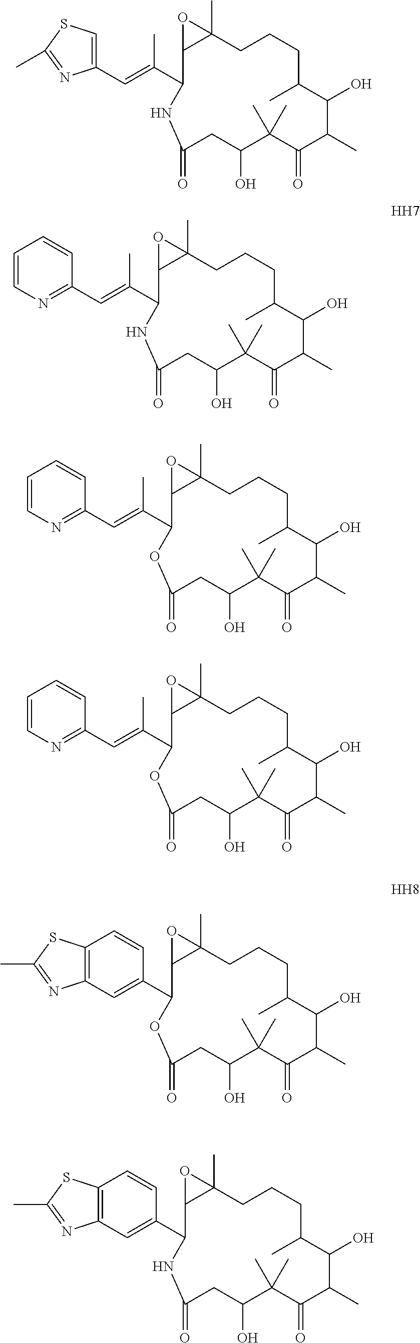

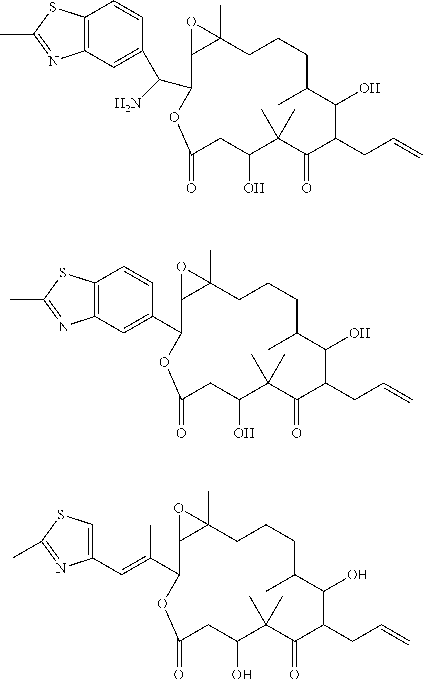

8. A compound of the following general formula VI: 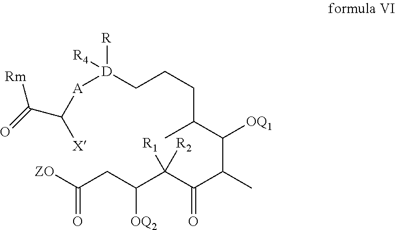
wherein
X′ is NHR₁₅, NR₁₅P, OH or OQ; wherein, R₁₅ is H, a methoxy group or an alkyl group; P is a N-protecting group;
Z is H, a substituted or unsubstituted alkyl group, a cycloalkyl group, an aryl group or a carboxyl-protecting group;
Q₁ and Q₂ each independently represents H, a C₁₋₄ alkyl group, NH₂ or a hydroxy-protecting group.

9. A pharmaceutical composition, wherein said pharmaceutical composition comprises the compound of claim 1, or a pharmaceutical acceptable salt, hydrate, polymorph, optical isomer, racemate, diastereomer or enantiomer thereof, and one or more pharmaceutical carriers and/or diluents.

10. The composition according to claim 9, wherein said composition further comprises one or more active agents.

11. A method for the treatment of a proliferative disease, comprising the administration of the compound of claim 1 to a subject in need thereof.

12. The method of claim 11 wherein said proliferative disease is selected from the group consisting of a tumor, multiple sclerosis, rheumatoid arthritis, atherosclerosis and restenosis.

13. A method for preparing the compound of claim 1, wherein, a 15-membered thiazole polyketide lactone compound or a 15-membered thiazole polyketide lactam compound is prepared from 14-hydroxy Epothilone by synthetic routes of reaction 2 to 4 in which a 15-membered macrocyclic lactonization or macrocyclic lactamization is performed;
compound L12 is obtained from Epothilone D and derivatives thereof LL according to synthetic route of reaction 5, which then reacts with phosphonium of formula L13 or L17 or L34 to perform 15-membered macrocyclic lactonization or lactamization to obtain compound L16 or L21 by synthetic route of reaction 5 and 6 or compound HH8; or
compound L32 is prepared by total synthesis as shown in synthetic route of reaction 12 and 12B by using compound L31-A, L31-B (L12).

14. A pharmaceutical composition, wherein said pharmaceutical composition comprises the compound of claim 3, or a pharmaceutical acceptable salt, hydrate, polymorph, optical isomer, racemate, diastereomer or enantiomer thereof, and one or more pharmaceutical carriers and/or diluents.

15. A pharmaceutical composition, wherein said pharmaceutical composition comprises the compound of claim 4, or a pharmaceutical acceptable salt, hydrate, polymorph, optical isomer, racemate, diastereomer or enantiomer thereof, and one or more pharmaceutical carriers and/or diluents.

16. A pharmaceutical composition, wherein said pharmaceutical composition comprises the compound of claim 5, or a pharmaceutical acceptable salt, hydrate, polymorph, optical isomer, racemate, diastereomer or enantiomer thereof, and one or more pharmaceutical carriers and/or diluents.

17. A pharmaceutical composition, wherein said pharmaceutical composition comprises the compound of claim 6, or a pharmaceutical acceptable salt, hydrate, polymorph, optical isomer, racemate, diastereomer or enantiomer thereof, and one or more pharmaceutical carriers and/or diluents.

18. A pharmaceutical composition, wherein said pharmaceutical composition comprises the compound of claim 7, or a pharmaceutical acceptable salt, hydrate, polymorph, optical isomer, racemate, diastereomer or enantiomer thereof, and one or more pharmaceutical carriers and/or diluents.

19. The composition according to claim 10, wherein said active agent is an anti-tumor or anti-cancer agent.

20. The method of claim 12, wherein said compound is used in combination with one or more anti-tumor or anti-cancer active agents.
